(19) United States
(12) Patent Application Publication
(10) Pub. No.: US 2019/0150805 A1
(43) Pub. Date: May 23, 2019

(54) BIOCOMPATIBLE IMPLANTABLE SENSOR APPARATUS AND METHODS

(71) Applicant: GlySens Incorporated, San Diego, CA (US)

(72) Inventors: Timothy Routh, San Diego, CA (US); Joseph Lucisano, San Diego, CA (US); William Markle, Mission Viejo, CA (US); Michael Perkins, Los Gatos, CA (US)

(21) Appl. No.: 16/253,095

(22) Filed: Jan. 21, 2019

Related U.S. Application Data
(62) Division of application No. 15/368,436, filed on Dec. 2, 2016.

(51) Int. Cl. A61B 5/145; A61B 5/00; A61B 5/1495; A61B 5/07

(52) U.S. Cl. CPC A61B 5/14503; A61B 5/6833; A61B 5/14532; A61B 5/14542; A61B 5/1495; A61B 5/7445; A61B 2560/0223; A61B 5/6861; A61B 5/741; A61B 5/7415; A61B 5/682; A61B 2560/0219; A61B 5/076

(57) ABSTRACT

Receiver apparatus for use with an analyte sensor, and methods of operation and manufacturing. In one embodiment, the analyte sensor is an implanted/implantable blood glucose sensor, including oxygen-based detector elements. The receiver apparatus is a wireless-enabled small form-factor device with limited functionality that can be easily worn or kept with the user on a continual basis, thereby obviating the need for a more fully featured receiver or smartphone for extended periods of time (e.g., one week). The exemplary oxygen based analyte sensor, with high degree of stability over time, enables the user to divorce themselves from the more fully functioned receiver or smartphone, since no external calibration of the sensor is required during the extended period. In one variant, the device is a lightweight wristband. Other variants include e.g., pendants, finger-worn rings, arm or head bands, skin patches, and even dental, subcutaneous, or prosthetic implants.

### RELATED APPLICATIONS

This application is related to co-owned and co-pending U.S. patent application Ser. No. 13/559,475 filed Jul. 26, 2012 entitled “Tissue Implantable Sensor With Hermetically Sealed Housing,” U.S. patent Ser. No. 14/982,346 filed Dec. 29, 2015 and entitled “Implantable Sensor Apparatus and Methods”, Ser. No. 15/170,571 filed Jun. 1, 2016 and entitled “Biocompatible Implantable Sensor Apparatus And Methods”, Ser. No. 15/197,104 filed Jun. 29, 2016 and entitled “Bio-adaptable Implantable Sensor Apparatus And Methods”, and Ser. No. 15/359,406 filed Nov. 22, 2016 and entitled “Heterogeneous Analyte Sensor Apparatus and Methods”, each of the foregoing incorporated herein by reference in its entirety. This application is also related to U.S. patent application Ser. No. 10/719,541 filed Nov. 20, 2003, now issued as U.S. Pat. No. 7,336,984 and entitled “Membrane and Electrode Structure for Implantable Sensor,” also incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### 1. TECHNICAL FIELD

The disclosure relates generally to the field of sensors, therapy devices, implants and other devices (such as those which can be used consistent with human beings or other living entities for in vivo detection and measurement or delivery of various solutes), and in one exemplary aspect to methods and apparatus enabling the use of such sensors and/or electronic devices for, e.g. monitoring of one or more physiological parameters, including through use of external receiver and processing apparatus.

### 2. DESCRIPTION OF RELATED TECHNOLOGY

Implantable electronics is a rapidly expanding discipline within the medical arts. Owing in part to significant advances in electronics and wireless technology integration, miniaturization, performance, and material biocompatibility, sensors or other types of electronics which once were beyond the realm of reasonable use in vivo in a living subject can now be surgically implanted within such subjects with minimal effect on the recipient subject, and in fact many inherent benefits.

One particular area of note relates to blood glucose monitoring for subjects, including those with so-called “type 1” or “type 2” diabetes. As is well known, regulation of blood glucose is impaired in people with diabetes by: (1) the inability of the pancreas to adequately produce the glucose-regulating hormone insulin; (2) the insensitivity of various tissues that use insulin to take up glucose; or (3) a combination of both of these phenomena. Safe and effective correction of this dysregulation requires blood glucose monitoring.

Currently, glucose monitoring in the diabetic population is based largely on collecting blood by “fingersticking” and determining its glucose concentration by conventional assay. This procedure has several disadvantages, including: (1) the discomfort associated with the procedure, which should be performed repeatedly each day; (2) the near impossibility of sufficiently frequent sampling (some blood glucose excursions require sampling every 20 minutes, or more frequently, to accurately treat); and (3) the requirement that the user initiate blood collection, which precludes warning strategies that rely on automatic early detection. Using the extant fingersticking procedure, the frequent sampling regimen that would be most medically beneficial cannot be realistically expected of even the most committed patients, and automatic sampling, which would be especially useful during periods of sleep, is not available.

Implantable glucose sensors have long been considered as an alternative to intermittent monitoring of blood glucose levels by the fingerstick method of sample collection. These devices may be fully implanted, where all components of the system reside within the body and there are no through-the-skin (i.e. percutaneous) elements, or they may be partially implanted, where certain components reside within the body but are physically connected to additional components external to the body via one or more percutaneous elements.

Accuracy is an important consideration for such implanted analyte sensors, especially in the context of blood glucose monitoring. Ensuring accurate measurement for extended periods of time (and minimizing the need for any other confirmatory or similar analyses) is of great significance.

FIG. 1 is a block diagram illustrating one exemplary prior art percutaneous blood glucose monitoring system 100. Exemplary of this class of devices is the Dexcom G5® Mobile CGM System. Manufactured by Dexcom Corporation, the Dexcom G5 system comprises a device 102 worn on the subject's outer abdomen 101, the device 102 which includes a small, transcutaneous probe (sensor needle) 103 with peroxide-based detector element 105, and detector circuitry 104 with wireless transmitter (i.e., Bluetooth-enabled device) 106. The transmitter 106 communicates wirelessly with a receiver/display device 108 which can be either (i) a user's smartphone or other personal electronic device with the Dexcom software “app” installed thereon, or (ii) a Dexcom G5 mobile receiver device (i.e., dedicated receiver). With the Dexcom G5 system, the user has access (via interface of the receiver or smartphone running the mobile app to a LAN/WAN 121) to a cloud-based reporting system (Dexcom CLARITY®), which ostensibly enables access and tracking of the user's glucose data via a web-based platform.

Devices such as the Dexcom G5 utilize peroxide-based blood glucose sensing, including a requirement for frequent external calibration (i.e., utilizing a separate confirmatory test such as a fingerstick). Per the manufacturer, the device should be calibrated at least once every twelve (12) hours via fingerstick or blood glucose (BG) meter, thereby making the device somewhat burdensome for the user in everyday operation (aside from issues associated with the transcutaneous sensor probe 103 and the associated requirement to continuously wear the device 102 externally on the abdomen).

Moreover, the receiver apparatus 108 must be kept in wireless proximity of the user (and the device 102) at all times; without the external receiver 108 and its display/alert features, the user has no way of being informed of their blood glucose level at any given time (or potentially dangerous situations such as rapid drops or increases in blood glucose level). While the maximum wireless range of the exemplary G5 device is generally commensurate with that of other Bluetooth-enabled devices (e.g., typically on the order of 30 feet or so), and hence gives some degree of flexibility to receiver placement relative to the user (and the sensor device 102), there are significant disabilities with this scheme, including notably the inability for the user to engage in some activities which require dissociation of the user (and device 102) from the receiver due to distance, an intervening and interfering medium such as water, etc., or incompatibility of the receiver with such media. Moreover, such receivers can add significant weight and/or bulk to the user when affixed thereto, thereby potentially reducing their competitiveness in high-end sporting activities such as marathons, gymnastics, triathlons, bicycle racing, etc.

Hence, in essence, a user of such prior art systems must have their receiver (dedicated or smartphone) with them at all times, and the system is unforgiving for transgressions of this requirement. For instance, a user leaving for work in the morning who forgets their smartphone or receiver at home has little choice but to turn around and retrieve it (unless they happen to keep a “spare” at the office).

Potential failure of such equipment (whether due to equipment failure, loss of battery charge, etc.) also requires some degree of “planning ahead” for the user including e.g., making sure that their receiver or smartphone is charged sufficiently, and/or a backup exists in case their receiver/smartphone is damaged or otherwise disabled.

The Assignee of the present disclosure has more recently developed improved methods and apparatus for implanting a blood glucose sensor (and measuring blood glucose level using the implanted sensor), which overcome the aforementioned disabilities with the prior art; see, inter alia, U.S. patent application Ser. Nos. 13/559,475, 14/982,346, 15/170,571, 15/197,104, and 15/359,406 previously incorporated herein.

However, Assignee has further recognized that one area of significant potential improvement relates to the external device “burdens” placed on a user, described above; i.e., making any external apparatus that the user must possess or utilize, and with which the user must interface, as minimal and user-transparent as possible, and reliable as possible, is highly desirable.

Moreover, user body (self) image, and perception of the user by others, are each of great significance to some individuals. Generally speaking, the less salient and more unobtrusive such users' blood analyte monitoring apparatus (and communications emanating therefrom) are, the more likely such users will continue to utilize the apparatus for their monitoring needs, and the less likely they will be to feel inhibited from participating in certain social situations.

### SUMMARY

The present disclosure satisfies the foregoing needs by providing, inter alia, improved apparatus for receiving sensed analyte levels within a living subject, including for extended periods of time without access to a smartphone or other comparable device, and methods of manufacturing and operating the same.

In one aspect of the disclosure, an apparatus for use with an implantable blood analyte sensing device is disclosed. In one embodiment, the apparatus includes a small form-factor electronic device which can be unobtrusively worn by the user on a continuous or near-continuous basis, and which can at least: (i) wirelessly receive signals from the implanted sensing device, and (ii) generate an output cognizable to the user relating to the sensed analyte level.

In one variant, the apparatus comprises an environmentally and mechanically robust wrist-band configured to generate a visual output (e.g., digital representation of blood glucose level in mg/dL or mmol/L). Alternatively, the apparatus may take the form of a neck pendant, badge or patch that can be temporarily affixed to the user's clothing, hair accessory, eyeglass/sunglass frame, or yet other personal accessory.

In another variant, the apparatus communicates with the user via a haptic device in contact with their skin, so as to e.g., indicate alerts, or even encode the blood glucose levels via haptic output.

In one implementation, the apparatus is configured to appear generally consistent with the appearance of a similar extant device (e.g., sports or cross-fit type wrist-worn physiological monitor), so as to appear to others that the user is merely wearing the extant device, versus a blood analyte data receiver.

In another implementation, the blood analyte display/haptic functionality is merely incorporated into the extant device, such as via a firmware upgrade and inclusion of an appropriate wireless interface and processing logic, or added onto the extant device via an add-on module (e.g., which snaps onto or adheres to the extant device).

In a further implementation, the apparatus includes a band (e.g., wrist band) for retention of the apparatus on the user, the band also comprising one or more radio frequency antenna elements therein.

In a further variant, the apparatus comprises an ear bud or ear plug which communicates with the user via audible output. In one implementation, the ear bud or plug is configured for wireless data communication with the implanted sensor and is battery powered, and the audible output comprises a synthesized voice readout of the numerical value of blood glucose level or other information of interest. In another implementation, the audible output comprises a series of discrete tones which encode the numeric value, and/or which are indicative of one or more alerts or action items for the host.

In yet another variant, the apparatus includes a substantially flexible patch which can be adhered to e.g., the user's skin. The patch in one implementation includes electronic circuitry printed on a substrate of the patch, and miniature integrated circuit (IC) devices embedded therein, as well as a flat or flexible LED (e.g., graphene-based), AMOLED, or OTFT (organic thin-film transistor) display device which is configured to display desired information such as analyte concentration in the wearer's blood based on received signals transmitted from the implanted sensor. In an exemplary configuration, the patch is powered by a flexible triboelectric or “static electricity”-based generator, and is kept in a dormant state except when the user desires to observe the display, and/or when the wireless receiver of the patch must be powered on to receive modulated RF signals from the implanted sensor.

In yet another variant, the apparatus includes a passively powered (i.e., by incident electromagnetic energy) patch which can be adhered to the subject's skin, clothing, etc., and which utilizes received RF energy from the implant transmitter (or another source) to demodulate the incoming RF signal, unscramble it, extract the data from the demodulated and unscrambled signal, process the extracted data, and cause illumination of one or more light sources (e.g., ultra-low power LEDs) on the patch indicating the estimated analyte level.

In one implementation of the foregoing “patch” variants, the patches are sold as a disposable commodity; e.g., a pack of twenty (20) which can be individually utilized by the user when a predecessor patch requires replacement due to loss, damage, or merely normal wear and tear.

In yet a further variant, the apparatus comprises an implant which is used to receive signals transmitted from the implanted analyte sensor, and produce an output indicative of analyte level cognizable by the host. In one implementation, the implant comprises a dental implant with radio frequency receiver and an acoustic transducer, and is configured to receive RF transmissions from the implanted sensor at a prescribed frequency, demodulate and extract sensed analyte data, process the data, and generate a host-audible output relating to the analyte level (e.g., via transmission to the host's auditory system via the host's jawbone).

In another aspect of the disclosure, a method of operating a blood analyte sensing system is disclosed. In one embodiment, the system includes an implantable analyte sensor, and an external reduced-capability receiver apparatus, and the method includes utilizing only the receiver apparatus for informing the host of the measured analyte level for a prescribed period of time, without resort to any other external calibration mechanism or input (whether via the receiver apparatus or otherwise). In one variant, the prescribed period of time comprises one (1) week.

In a further aspect, apparatus for use with an implanted blood analyte sensing device is disclosed. In one embodiment, the apparatus includes: wireless receiver apparatus configured to receive wireless signals from the blood analyte sensing device, the wireless signals encoding data relating to levels of the blood analyte; data processing apparatus in data communication with the wireless receiver apparatus and configured to utilize the encoded data to determine a blood analyte level; an electrical power source configured to supply electrical power; and indicator apparatus in communication with the data processing apparatus and electrical power source, the indicator apparatus configured to indicate to a user the determined blood analyte level.

In one variant, the implanted blood analyte sensing device includes at least one oxygen-based sensor, and the apparatus is configured to operate without communication with a parent platform or external calibration for at least a prescribed period of time (e.g., one week).

In another variant, the wireless signals encoding data are scrambled according to a unique scrambling code prior to transmission from the device, and the apparatus is further configured to unscramble the received wireless signals based at least in part on the unique scrambling code. The wireless signals are transmitted from the device e.g., only at prescribed times or prescribed intervals, and the apparatus is configured to enable the wireless receiver apparatus to receive the wireless signals only during the prescribed times or at the prescribed intervals, and otherwise maintain at least a portion of the wireless receiver apparatus in a dormant or sleep state so as to conserve electrical power of the electrical power source.

In a further variant, the wireless signals are transmitted from the device only at prescribed times, and the apparatus is configured to enable the wireless receiver apparatus to receive the wireless signals during a number n of the prescribed times, the number n less than a total number of transmissions of the wireless signals, the apparatus configured to dynamically vary the number n based at least on one or more operational parameters, such as e.g., a remaining level of power in the electrical power source, a time period from when a last prior calibration was applied to the data relating to levels of the blood analyte, or the determined blood analyte level, and/or detection of an ambulatory or non-ambulatory state of the user.

In another variant, the apparatus is further configured to utilize at least the data relating to levels of the blood analyte to determine at least one of: (i) a trend, and/or (ii) a rate of change of blood analyte level; and the one or more operational parameters includes the at least one of the determined (i) trend or (ii) rate of change.

In yet a further variant, the one or more operational parameters includes a proximity to a prescribed boundary or warning criterion associated with determined blood analyte level.

In one implementation, the apparatus includes a small form-factor wearable apparatus, such as a wrist-worn apparatus, pendant, fob, wrist or arm band, etc. In one configuration, the wrist worn apparatus is configured to indicate via the indicator apparatus only a prescribed subset of values, each value of the prescribed subset determined from the received wireless signals only. The prescribed subset of values may include for example: (i) the determined blood analyte level; (ii) a blood analyte level trend indication; and (iii) a blood analyte level rate of change indication.

In another implementation, the wrist-worn apparatus comprising a primary function, and the use with the implanted blood analyte sensing device comprises a secondary function; and the data processing apparatus and the indicator apparatus are configured to execute both the primary function and the secondary function(s). The secondary (e.g., glucose monitoring) function(s) may be enabled through e.g., at least one of a software and/or firmware download to the apparatus after its manufacture. For instance, an NFC- and Bluetooth-enabled smart watch may also have the blood analyte monitoring functions “piggy-backed” onto its normal functions.

In another variant, the apparatus further includes a wireless transceiver apparatus in data communication with the data processing apparatus; the wireless receiver comprises a narrowband radio frequency (RF) receiver; and the wireless transceiver includes a personal area network (PAN) RF transceiver configured to operate within a frequency range which does not overlap with the narrowband RF. In one implementation, the apparatus is further configured to opportunistically establish a communication session with a parent platform via the PAN RF transceiver when such parent platform is at least one of: (i) within sufficient range to establish the communication session; and/or (ii) has sufficient signal strength at the apparatus to establish the communication session. Upon establishment of the communication session, transfer at least one of calibration data and/or configuration data from the parent platform to the apparatus for use by the apparatus until a subsequent opportunistic communication session is established.

In yet another variant, the apparatus comprises a small form-factor wearable apparatus configured to maintain contact with a user's skin, and the indicator apparatus comprises a haptic output apparatus configured to generate haptic output cognizable by the user via the contact, the haptic output configured to encode at least one of: (i) the determined blood analyte level; and/or (ii) one or more alerts or alarms.

In one implementation, the electrical power source comprises a triboelectric generation apparatus. In another implementation, the electrical power source comprises a thermoelectric or Seebeck Effect generation apparatus. In yet another implementation, the electrical power source comprises a solar or photoelectric effect power generation apparatus.

In a further variant, the apparatus further includes a substantially flexible non-conductive substrate with a plurality of electrical traces disposed thereon; and a skin-adherent material configured to enable adherence of the apparatus to a skin of the user such that the substrate at least partly flexes out of a planar geometry as part of the adherence. The indicator apparatus includes a plurality of light-emitting diodes (LEDs) such as OLEDs, configured to generate a numeric indication when illuminated corresponding to the determined blood analyte level. In one implementation, the apparatus is configured to be used for only a prescribed duration, and disposable thereafter.

In still a further variant, the apparatus is configured to be implanted within the user, such as sub-dermally, or within a tooth or other dental structure (e.g., crown, bridge, etc.) of the user. In one implementation, the dental implant includes a transducer configured to generate vibrations that can be transmitted to at least one ear of the user via at least a jawbone of the user. In one configuration, the generated vibrations comprise vibrations forming at least one audible tone within an audible range of a human (20 Hz to 20 KHz nominal), the at least one tone encoding information relating to the determined blood analyte level. In another configuration, the indicator apparatus further includes a speech synthesis apparatus, and the generated vibrations comprise synthesized speech communications within a range of 20 Hz to 20 KHz, the synthesized speech comprising a speech representation of the determined blood analyte level.

In another aspect of the disclosure, a method of operating a blood analyte evaluation system is disclosed. In one embodiment, the system includes an implantable sensor, a local receiver, and a parent platform, and the method includes: utilizing the local receiver to receive and process wireless data transmitted from the implantable sensor on a substantially continuous basis during a first period of time, the local receiver not having data communication with the parent platform during the first period; and only incidentally establishing communication between the local receiver and the parent platform to at least receive calibration data at the local receiver from the parent platform.

In one variant, the incidentally establishing communication between the local receiver and the parent platform includes establishing communication after the first period, and the method further includes utilizing the received calibration data to perform at least one of: (i) confirmation of a current calibration of the implantable sensor; and/or (ii) adjustment to a current calibration of the implantable sensor.

In one implementation, the utilizing the local receiver to receive wireless data transmitted from the implantable sensor includes utilizing a dedicated narrowband wireless receiver to receive the wireless data; and communication between the local receiver and the parent platform includes use of a multiband wireless transceiver apparatus, one or more frequencies of the dedicated narrowband receiver not overlapping with a frequency range utilized by the multiband transceiver apparatus.

In another variant, the only incidental communication between the local receiver and the parent platform to at least receive calibration data at the local receiver from the parent platform further includes receiving user-provided configuration data at the local receiver from the parent platform; and the method further includes utilizing the received configuration data to configure at least one aspect of a user indicator function of the local receiver.

In a further variant, the processing includes determination of a blood glucose level, and the method further includes causing providing to a user within which the implantable sensor is implanted, during the period, one or more representations of the determined blood glucose level.

In yet another aspect of the disclosure, apparatus for use with an implanted blood glucose sensing device is disclosed. In one embodiment, the apparatus is configured to generate an indication cognizable by a user and representative of the user's blood glucose level according to the method comprising: receiving at a power generation device of the apparatus incident first electromagnetic energy; converting the received electromagnetic energy to electrical power; providing the electrical power to at least a processing device of the apparatus and a wireless receiver of the apparatus; receiving at the wireless receiver a plurality of wireless signals generated by the sensing device and encoding data relating to the blood glucose level; processing the received plurality of signals using the processing device to generate an estimate of the blood glucose level; and utilizing an indicator apparatus in communication with the processing device to generate the indication.

In one variant, the apparatus comprises a skin-adherable patch form factor, and the incident first electromagnetic energy comprises solar radiation. In another variant, the incident first electromagnetic energy comprises electromagnetic energy emitted by the implanted sensing device (e.g., the plurality of wireless signals, such as ones emitted predominantly at a prescribed center frequency within a range comprising 400 MHz to 450 MHz inclusive).

In another aspect of the disclosure, a method of monitoring blood glucose level while engaging in a sporting activity is disclosed. In one embodiment, the method includes wearing a limited functionality and limited form-factor local wireless receiver apparatus to: (i) receive data wirelessly transmitted from an implanted blood glucose sensor; (ii) process the received data to generate at least an estimated blood glucose level; and (iii) provide indication of the estimated blood glucose level to the user during the sporting activity. In one variant, the limited form factor is enabled at least in part by the limited functionality (i.e., less functionality equates to smaller form factor), and the limited form factor enhances or enables performance of the sporting activity (e.g., reduces overall wearer weight, hydrodynamic friction, bulk, etc.).

In one implementation, the limited functionality includes indication via an indicator apparatus of the local wireless receiver apparatus of only a prescribed subset of values, each value of the prescribed subset determined from the received wirelessly transmitted data only; e.g., (i) the estimated blood glucose level; (ii) a blood glucose level trend indication; and (iii) a blood glucose level rate of change indication.

In another aspect, a blood glucose monitoring patch for use on a living being is disclosed. In one embodiment, the patch includes: a wireless receiver apparatus; data processor apparatus in signal communication with the wireless receiver apparatus; indicator apparatus in communication with the data processing apparatus; and a power supply configured to supply electrical power to at least the wireless receiver apparatus, the data processor apparatus, and the indicator apparatus. In one variant, the wireless receiver apparatus, data processor apparatus, and indicator apparatus cooperate to (i) enable reception of wirelessly transmitted data from a sensor implanted in the living being, (ii) enable processing of the received data to produce an estimated blood glucose level, and (iii) cause indication of the estimated blood glucose level to the user.

In another variant, the patch is at least partly flexible and is configured to adhere to the skin of the living being for at least a period of time without removal. The power supply comprises a triboelectric generator apparatus that obviates use of a replaceable battery.

In another variant, the patch is configured to be disposable, and the period of time comprises at least one (1) week.

In a further variant, the patch is configured to operate during the period of time, including the production of the estimated blood glucose level and indication thereof, without confirmation or calibration.

Other features and advantages of the present disclosure will immediately be recognized by persons of ordinary skill in the art with reference to the attached drawings and detailed description of exemplary embodiments as given below.

### BRIEF DESCRIPTION OF THE DRAWINGS

FIG. 1 is a logical block diagram illustrating a typical prior art transcutaneous blood analyte monitoring system with external receiver.

FIG. 2 is a front perspective view of one exemplary embodiment of a fully implantable biocompatible sensor apparatus useful with various aspects of the present disclosure.

FIGS. 2A-2C are top, bottom, and side elevation views, respectively, of the exemplary sensor apparatus of FIG. 2.

FIG. 3 is a logical block diagram illustrating one embodiment of a system architecture for, inter alia, monitoring blood analyte levels within a user, according to the present disclosure.

FIG. 3A is a logical block diagram illustrating another embodiment of a system architecture for, inter alia, monitoring blood analyte levels within a user, according to the present disclosure.

FIG. 3B is a logical block diagram illustrating yet another embodiment of a system architecture for, inter alia, monitoring blood analyte levels within a user, according to the present disclosure.

FIG. 3C is a functional block diagram illustrating an exemplary implantable sensor apparatus and local receiver apparatus according to one embodiment of the present disclosure.

FIG. 4A is a functional block diagram illustrating an exemplary embodiment of the local receiver apparatus of FIG. 3C.

FIG. 4B is a functional block diagram illustrating another exemplary embodiment of the local receiver apparatus of FIG. 3C, wherein a biocompatible (e.g., implanted) output receiver is used in conjunction therewith.

FIG. 4C is a functional block diagram illustrating yet another exemplary embodiment of the local receiver apparatus of FIG. 3C, wherein an external output receiver is used in conjunction therewith.

FIG. 4D is a functional block diagram illustrating an exemplary embodiment of the output receiver of FIGS. 4B and 4C.

FIG. 4E is a functional block diagram illustrating yet a further exemplary embodiment of the local receiver apparatus of FIG. 3C, wherein the local receiver apparatus is implanted within a host and communicates wirelessly with both a blood analyte (e.g., glucose) sensor and a parent platform.

FIGS. 4F-1 through 4F-3 are top, side, and perspective elevation views of a first embodiment of a wearable local receiver apparatus according to the disclosure.

FIGS. 4G-1 through 4G-3 are top, side, and perspective elevation views of a second embodiment of a wearable local receiver apparatus according to the disclosure.

FIGS. 4H-1 through 4H-3 are top, side, and perspective elevation views of a third embodiment of a wearable local receiver apparatus according to the disclosure.

FIGS. 4I-1 through 4I-3 are top, side, and perspective elevation views of a fourth embodiment of a wearable local receiver apparatus according to the disclosure.

FIGS. 4J-1 through 4J-3 are top, side, and perspective elevation views of a fifth embodiment of a wearable local receiver apparatus according to the disclosure.

FIGS. 4K-1 through 4K-3 are top, side, and perspective elevation views of a sixth embodiment of a wearable local receiver apparatus according to the disclosure.

FIGS. 4L-1 through 4L-3 are top, side, and perspective elevation views of a seventh embodiment of a wearable local receiver apparatus according to the disclosure.

FIGS. 4M-1 and 4M-2 are top and bottom perspective views, respectively, of another embodiment of the local receiver apparatus of the disclosure, configured for use in a pendant or fob form factor.

FIG. 4N is a front and side plan view of another embodiment of a user-wearable local receiver apparatus, configured as a flexible skin-adherent patch.

FIG. 4O is a side cross-sectional view of an implantable local receiver apparatus, configured as a dental implant.

FIG. 5 is a logical flow diagram illustrating one exemplary embodiment of a method of operating a local receiving device for blood analyte measurement according to the present disclosure.

FIG. 5A is a logical flow diagram illustrating one exemplary implementation of the sensor data processing and output according to the method of FIG. 5.

FIG. 5B is a logical flow diagram illustrating one exemplary implementation of the sensor data receipt and demodulation/unscrambling according to the method of FIG. 5A.

All Figures © Copyright 2016 GlySens Incorporated. All rights reserved.

### DETAILED DESCRIPTION

Reference is now made to the drawings, wherein like numerals refer to like parts throughout.

### Overview

One aspect of the present disclosure leverages Assignee's recognition that many of the above-described disabilities of the prior art “receiver” approach (including the user being effectively tethered to their analyte monitoring system receiver) can be mitigated or even completely eliminated.

Accordingly, the present disclosure makes use in one exemplary embodiment of a minimal profile (and functionality) receiving device which the user can discretely carry or wear continuously, so as to obviate the bulky and more full-featured receiver(s) of the prior art. In this fashion, the user is largely freed from concerns such as forgetting their receiver/smartphone, having it maintained in a constant state of charge, and notably refraining from activities which would otherwise be impossible or at least highly impractical under the prior art (e.g., watersports including swimming, surfing, scuba and other diving; combat sports including boxing and martial arts; fitness activities or certain competitive sports where a bulky receiver could be damaged or dislodged by vigorous physical activity; performance or other social activities where a bulky receiver would be inappropriate or distracting; or even limited duration space travel).

In one implementation, the aforementioned implantable sensor with oxygen-based detector element(s) is used in conjunction with a small form-factor, limited function wrist device which provides the user with a continuously wearable, all-environment device that outputs necessary user information (including in one variant a digital display of the user's blood glucose level in mg/dL or mmol/L and associated rate/trend indication). Hence, the user experience with the device and quality of life are improved through obviation of the need for contact between the implanted sensor and the user's smartphone or other such receiver (or cloud entity) for at least extended periods of time (e.g., days or even weeks).

In one variant, the aforementioned small form-factor device is battery operated and is configured for ultra-low power consumption, such that the user can wear and utilize the device for extended periods of time without a battery change or charge. Power conservation is accomplished in one configuration through use of one or more of: (i) non-continuous display; (ii) ambient light level sensing (and concurrent adjustment of LED or other display element intensity); and/or (iii) sleep modes for various non-essential components or portions thereof, and ultra-low voltage/low-power integrated circuits (ICs).

In another variant, the small form-factor device utilizes a haptic feedback apparatus either in place of or in conjunction with the aforementioned display so as to discretely inform or alert the user regarding information relating to blood glucose level. For instance, in one configuration, the haptic feedback apparatus is in contact with the user's skin (e.g., on the underside of the aforementioned wrist device) and is used to alert the user as to the need to check their blood glucose level using e.g., the display device, but without the more externally noticeable spontaneous activation of the LED or other display element. In this fashion, the user maintains complete discretion of the information with respect to others around him/her.

Other configurations for the small form-factor device described herein include (without limitation) pendants, jewelry (e.g., rings), dermal patches, portions of garments, wrist or arm bands, hair accessories (e.g., hair bands), eyeglasses, earplugs or other ear-worn accessories, and even implants (e.g., dental or sub-dermal implants) or portions of prosthetics.

Methods of use of the aforementioned membranes and sensor elements are also disclosed herein.

### Detailed Description of Exemplary Embodiments

Exemplary embodiments of the present disclosure are now described in detail. While these embodiments are primarily discussed in the context of a fully implantable glucose sensor, such as those exemplary embodiments described herein, and/or those set forth in U.S. Patent Application Publication No. 2013/0197332 filed Jul. 26, 2012 entitled “Tissue Implantable Sensor With Hermetically Sealed Housing;” U.S. Pat. No. 7,894,870 to Lucisano et al. issued Feb. 22, 2011 and entitled “Hermetic Implantable Sensor;” U.S. Patent Application Publication No. 2011/0137142 to Lucisano et al. published Jun. 9, 2011 and entitled “Hermetic Implantable Sensor;” U.S. Pat. No. 8,763,245 to Lucisano et al. issued Jul. 1, 2014 and entitled “Hermetic Feedthrough Assembly for Ceramic Body;” U.S. Patent Application Publication No. 2014/0309510 to Lucisano et al. published Oct. 16, 2014 and entitled “Hermetic Feedthrough Assembly for Ceramic Body;” U.S. Pat. No. 7,248,912 to Gough et al. issued Jul. 24, 2007 and entitled “Tissue Implantable Sensors for Measurement of Blood Solutes;” and U.S. Pat. No. 7,871,456 to Gough et al. issued Jan. 18, 2011 and entitled “Membranes with Controlled Permeability to Polar and Apolar Molecules in Solution and Methods of Making Same;” and U.S. Patent Application Publication No. 2013/0197332 to Lucisano et al. published Aug. 1, 2013 and entitled “Tissue Implantable Sensor with Hermetically Sealed Housing;” PCT Patent Application Publication No. 2013/016573 to Lucisano et al. published Jan. 31, 2013 and entitled “Tissue Implantable Sensor with Hermetically Sealed Housing,” each of the foregoing incorporated herein by reference in its entirety, as well as those of U.S. patent application Ser. Nos. 13/559,475, 14/982,346, 15/170,571, and 15/197,104 previously incorporated herein, it will be recognized by those of ordinary skill that the present disclosure is not so limited. In fact, the various aspects of the disclosure are useful with, inter alia, other types of implantable sensors and/or electronic devices.

Further, while the following embodiments describe specific implementations of e.g., biocompatible oxygen-based multi-sensor element devices for measurement of glucose, having specific configurations, protocols, locations, and orientations for implantation (e.g., proximate the waistline on a human abdomen with the sensor array disposed proximate to fascial tissue; see e.g., U.S. patent application Ser. No. 14/982,346 filed Dec. 29, 2015 and entitled “Implantable Sensor Apparatus and Methods” previously incorporated herein), those of ordinary skill in the related arts will readily appreciate that such descriptions are purely illustrative, and in fact the methods and apparatus described herein can be used consistent with, and without limitation: (i) in living beings other than humans; (ii) other types or configurations of sensors (e.g., other types, enzymes, and/or theories of operation of glucose sensors, sensors other than glucose sensors, such as e.g., sensors for other analytes such as urea, lactate); (iii) other implantation locations and/or techniques (including without limitation transcutaneous or non-implanted devices as applicable); and/or (iv) devices intended to deliver substances to the body (e.g. implanted drug pumps, drug-eluting solid materials, and encapsulated cell-based implants, etc.); and/or other devices (e.g., non-sensors and non-substance delivery devices).

As used herein, the term “analyte” refers without limitation to a substance or chemical species that is of interest in an analytical procedure. In general, the analyte itself cannot be measured, but a measurement of the analyte (e.g., glucose) can be derived through measurement of chemical constituents, components, or reaction byproducts associated with the analyte (e.g., hydrogen peroxide, oxygen, free electrons, etc.).

As used herein, the terms “biocompatible” and “biocompatibility” refer without limitation to the ability of a medical device or implantable material to perform as intended in the presence of an appropriate host wound healing response and/or other immunogenic responses, while minimizing magnitude and duration of the wound healing (e.g., acute inflammation, chronic inflammation, foreign body reaction (FBR), and fibrosis/fibrous capsule development) and causing no significant harm to the patient.

As used herein, the terms “detector” and “sensor” refer without limitation to a device having one or more elements (e.g., detector element, sensor element, sensing elements, etc.) that generate, or can be made to generate, a signal indicative of a measured parameter, such as the concentration of an analyte (e.g., glucose) or its associated chemical constituents and/or byproducts (e.g., hydrogen peroxide, oxygen, free electrons, etc.). Such a device may be based on electrochemical, electrical, optical, mechanical, thermal, or other principles as generally known in the art. Such a device may consist of one or more components, including for example, one, two, three, or four electrodes, and may further incorporate immobilized enzymes or other biological or physical components, such as membranes, to provide or enhance sensitivity or specificity for the analyte.

As used herein, the terms “orient,” “orientation,” and “position” refer, without limitation, to any spatial disposition of a device and/or any of its components relative to another object or being, and in no way connote an absolute frame of reference.

As used herein, the terms “top,” “bottom,” “side,” “up,” “down,” and the like merely connote, without limitation, a relative position or geometry of one component to another, and in no way connote an absolute frame of reference or any required orientation. For example, a “top” portion of a component may actually reside below a “bottom” portion when the component is mounted to another device (e.g., host sensor).

As used herein the term “parent platform” refers without limitation to any device, group of devices, and/or processes with which a client or peer device (including for example the various embodiments of local receiver described here) may logically and/or physically communicate to transfer or exchange data. Examples of parent platforms can include, without limitation, smartphones, tablet computers, laptops, smart watches, personal computers/desktops, servers (local or remote), gateways, dedicated or proprietary analyte receiver devices, medical diagnostic equipment, and even other local receivers acting in a peer-to-peer or dualistic (e.g., master/slave) modality.

As used herein, the term “application” (or “app”) refers generally and without limitation to a unit of executable software that implements a certain functionality or theme. The themes of applications vary broadly across any number of disciplines and functions (such as on-demand content management, e-commerce transactions, brokerage transactions, home entertainment, calculator etc.), and one application may have more than one theme. The unit of executable software generally runs in a predetermined environment; for example, the Java™ environment.

As used herein, the term “computer program” or “software” is meant to include any sequence or human or machine cognizable steps which perform a function. Such program may be rendered in virtually any programming language or environment including, for example, C/C++, Fortran, COBOL, PASCAL, assembly language, markup languages (e.g., HTML, SGML, XML, VoXML), and the like, as well as object-oriented environments such as the Common Object Request Broker Architecture (CORBA), Java™ (including J2ME, Java Beans, etc.) and the like.

As used herein, the terms “Internet” and “internet” are used interchangeably to refer to inter-networks including, without limitation, the Internet. Other common examples include but are not limited to: a network of external servers, “cloud” entities (such as memory or storage not local to a device, storage generally accessible at any time via a network connection, or cloud-based or distributed processing or other services), service nodes, access points, controller devices, client devices, etc.

As used herein, the term “memory” includes any type of integrated circuit or other storage device adapted for storing digital data including, without limitation, ROM, PROM, EEPROM, DRAM, SDRAM, DDR/2 SDRAM, EDO/FPMS, RLDRAM, SRAM, “flash” memory (e.g., NAND/NOR), 3D memory, and PSRAM.

As used herein, the terms “microprocessor” and “processor” or “digital processor” are meant generally to include all types of digital processing devices including, without limitation, digital signal processors (DSPs), reduced instruction set computers (RISC), general-purpose (CISC) processors, microprocessors, gate arrays (e.g., FPGAs), PLDs, reconfigurable computer fabrics (RCFs), array processors, secure microprocessors, and application-specific integrated circuits (ASICs). Such digital processors may be contained on a single unitary integrated circuit (IC) die, or distributed across multiple components.

As used herein, the term “network” refers generally to any type of telecommunications or data network including, without limitation, hybrid fiber coax (HFC) networks, satellite networks, telco networks, and data networks (including MANs, WANs, LANs, WLANs, internets, and intranets), cellular networks, as well as so-called “mesh” networks and “IoTs” (Internet(s) of Things). Such networks or portions thereof may utilize any one or more different topologies (e.g., ring, bus, star, loop, etc.), transmission media (e.g., wired/RF cable, RF wireless, millimeter wave, optical, etc.) and/or communications or networking protocols.

As used herein, the term “interface” refers to any signal or data interface with a component or network including, without limitation, those of the FireWire (e.g., FW400, FW800, etc.), USB (e.g., USB 2.0, 3.0. OTG), Ethernet (e.g., 10/100, 10/100/1000 (Gigabit Ethernet), 10-Gig-E, etc.), MoCA, LTE/LTE-A, Wi-Fi (802.11), WiMAX (802.16), Z-wave, PAN (e.g., 802.15)/Zigbee, Bluetooth, or power line carrier (PLC) families.

As used herein, the term “QAM” refers to modulation schemes used for data or signals. Such modulation scheme might use any constellation level (e.g. QPSK, 16-QAM, 64-QAM, 256-QAM, etc.).

As used herein, the term “server” refers to any computerized component, system or entity regardless of form which is adapted to provide data, files, applications, content, or other services to one or more other devices or entities on a computer network.

As used herein, the term “storage” refers to without limitation computer hard drives, memory, RAID devices or arrays, optical media (e.g., CD-ROMs, Laserdiscs, Blu-Ray, etc.), solid state devices (SSDs), flash drives, cloud-hosted storage, or network attached storage (NAS), or any other devices or media capable of storing data or other information.

As used herein, the term “Wi-Fi” refers to, without limitation and as applicable, any of the variants of IEEE-Std. 802.11 or related standards including 802.11 a/b/g/n/s/v/ac or 802.11-2012/2013, as well as Wi-Fi Direct (including inter alia, the “Wi-Fi Peer-to-Peer (P2P) Specification”, incorporated herein by reference in its entirety).

As used herein, the term “wireless” means any wireless signal, data, communication, or other interface including without limitation Wi-Fi, Bluetooth, 3G (3GPP/3GPP2), HSDPA/HSUPA, TDMA, CDMA (e.g., IS-95A, WCDMA, etc.), FHSS, DSSS, GSM, PAN/802.15, WiMAX (802.16), 802.20, Zigbee®, Z-wave, narrowband/FDMA, OFDM, PCS/DCS, LTE/LTE-A, analog cellular, CDPD, satellite systems, millimeter wave or microwave systems, acoustic, and infrared (i.e., IrDA).

### Exemplary Implantable Sensor

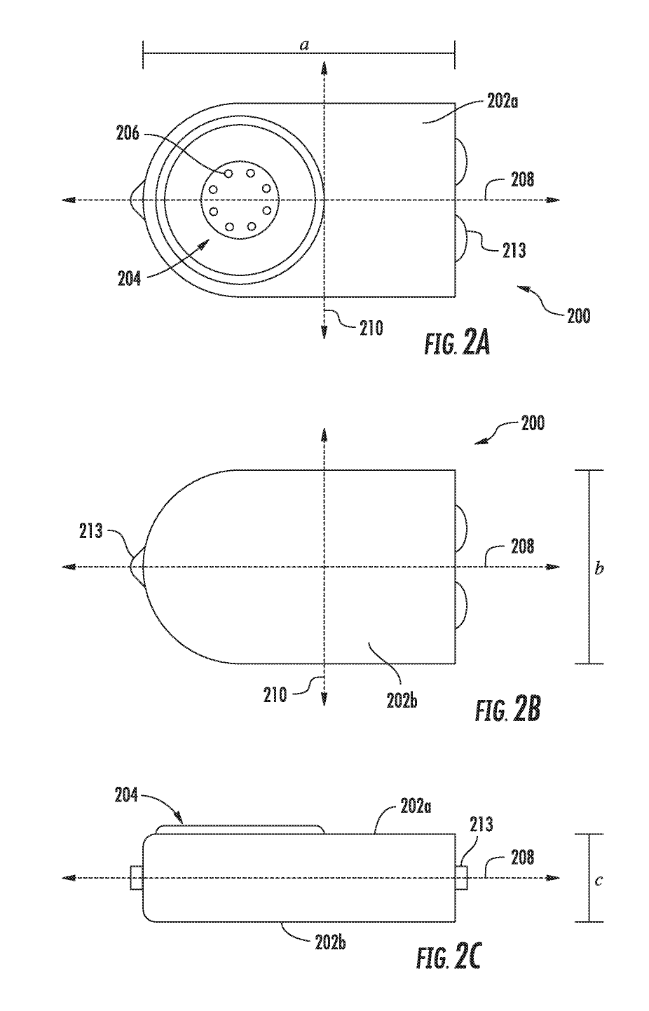

Referring now to FIGS. 2-2C, one exemplary embodiment of a sensor apparatus useful with various aspects of the present disclosure is shown and described.

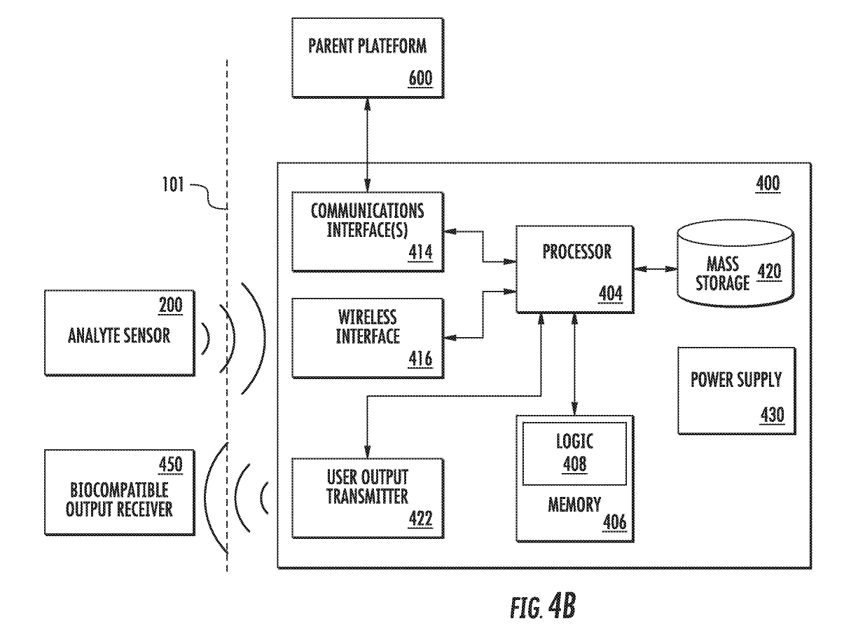

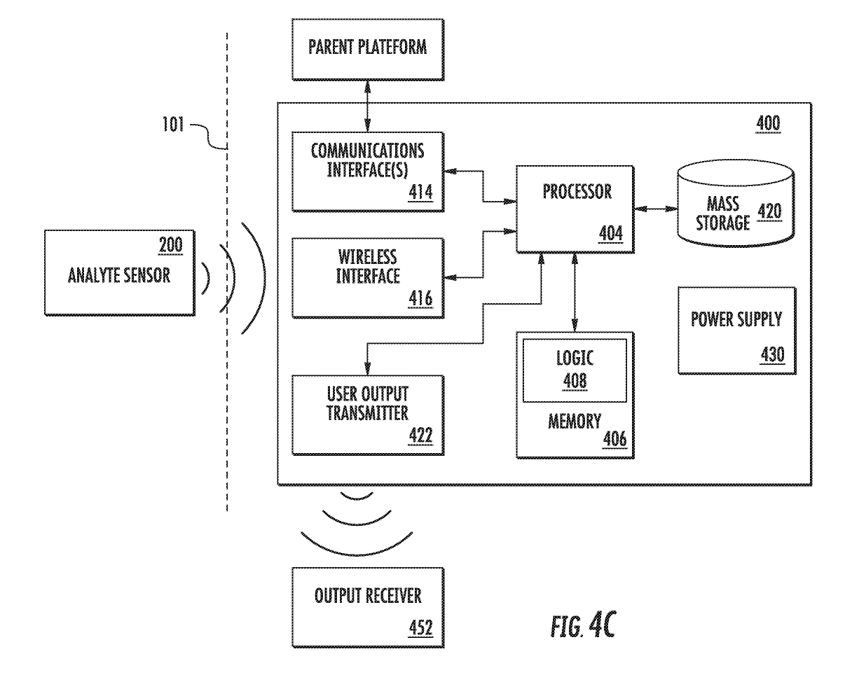

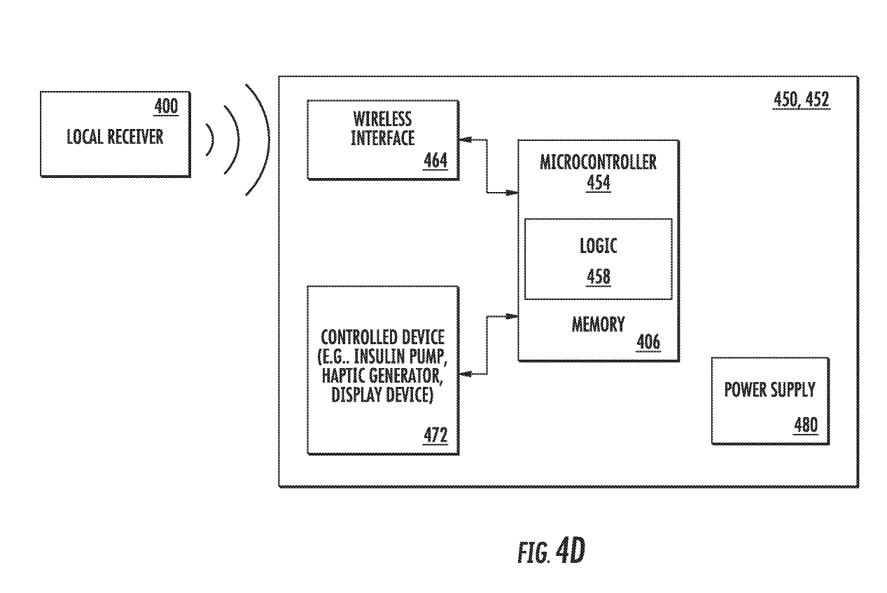

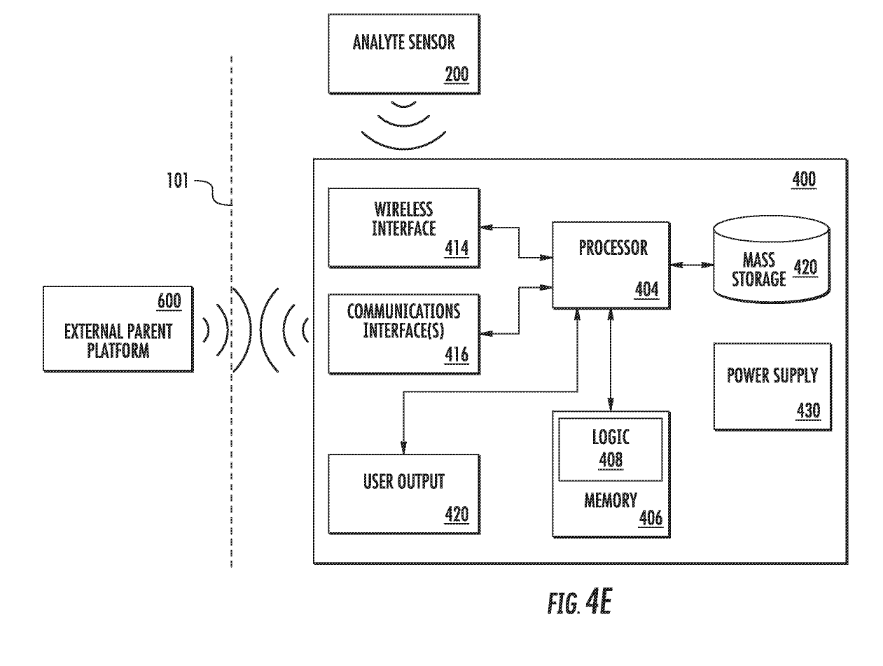

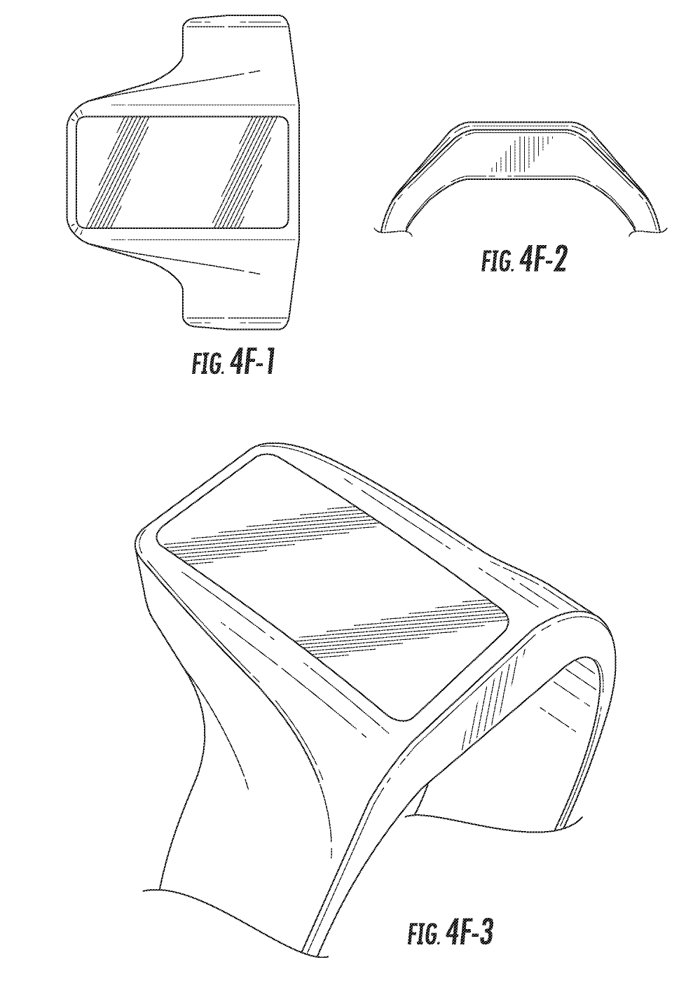

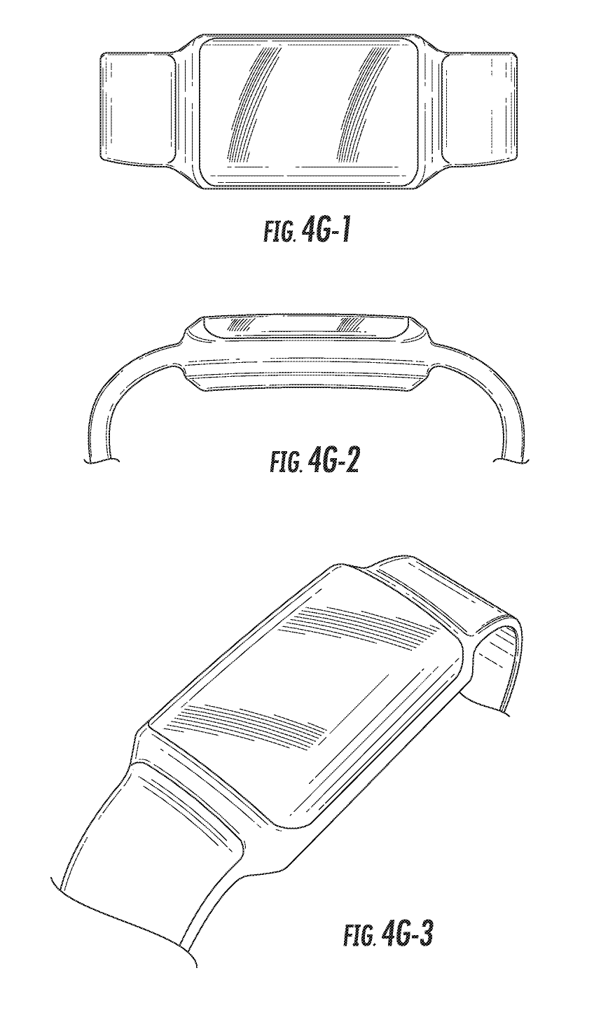

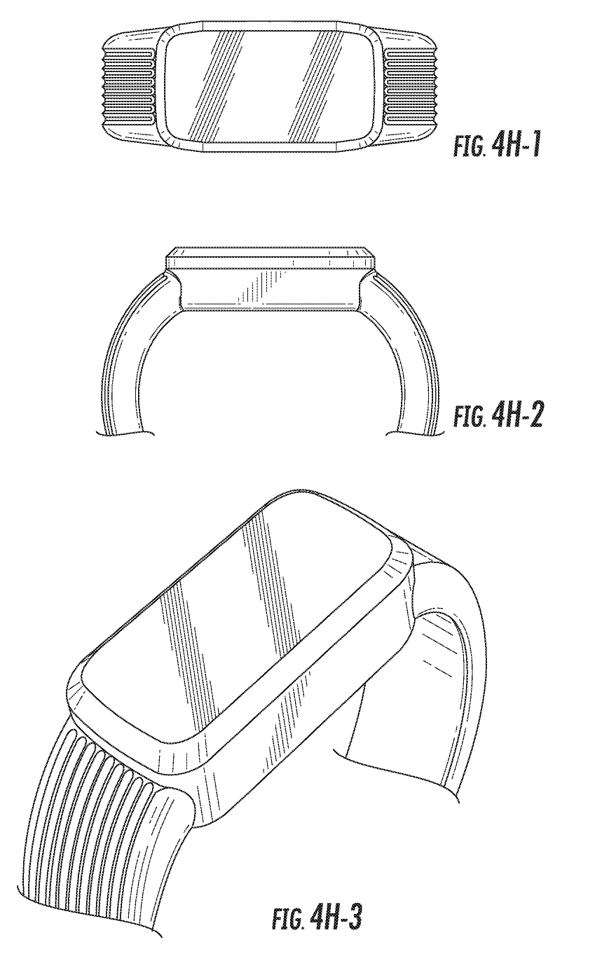

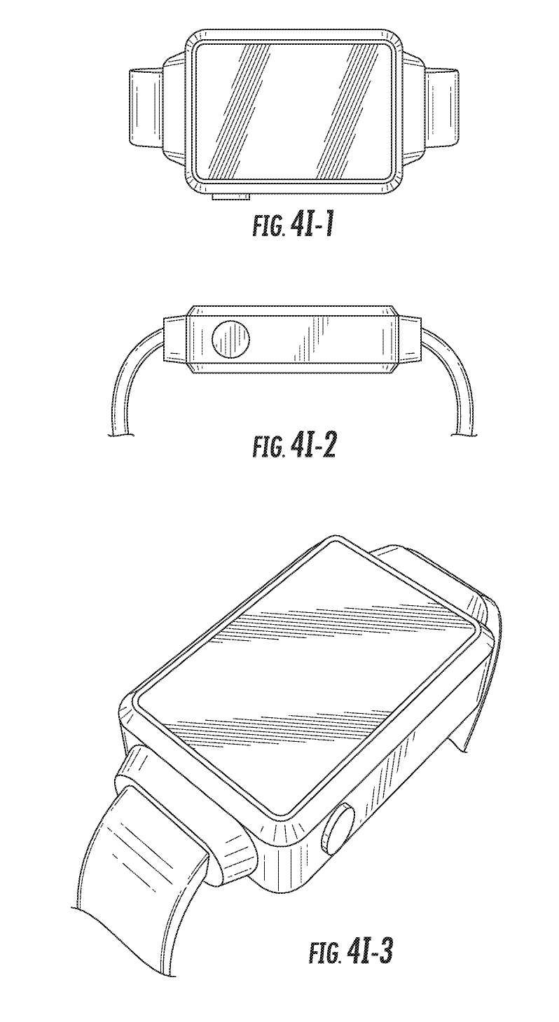

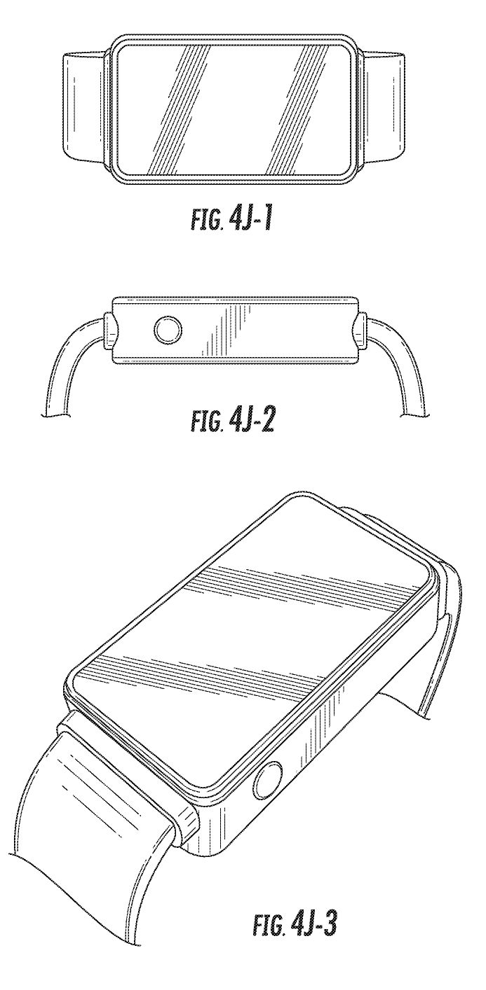

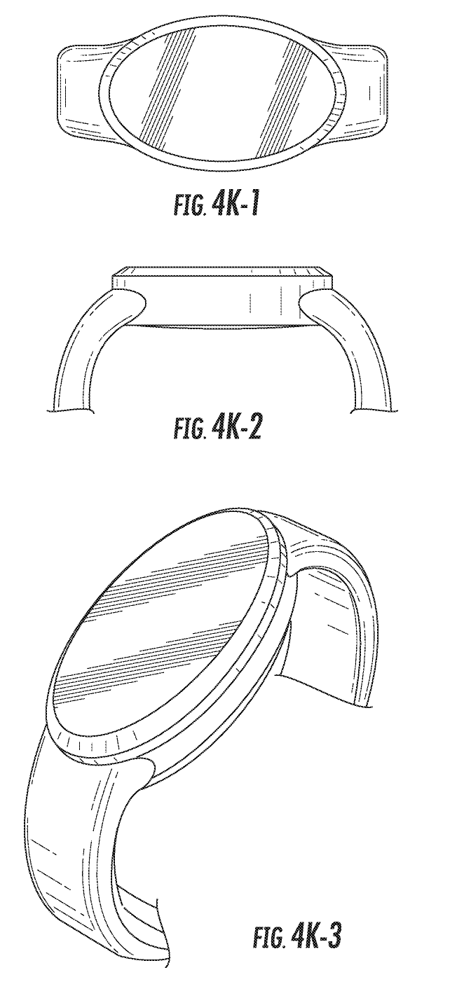

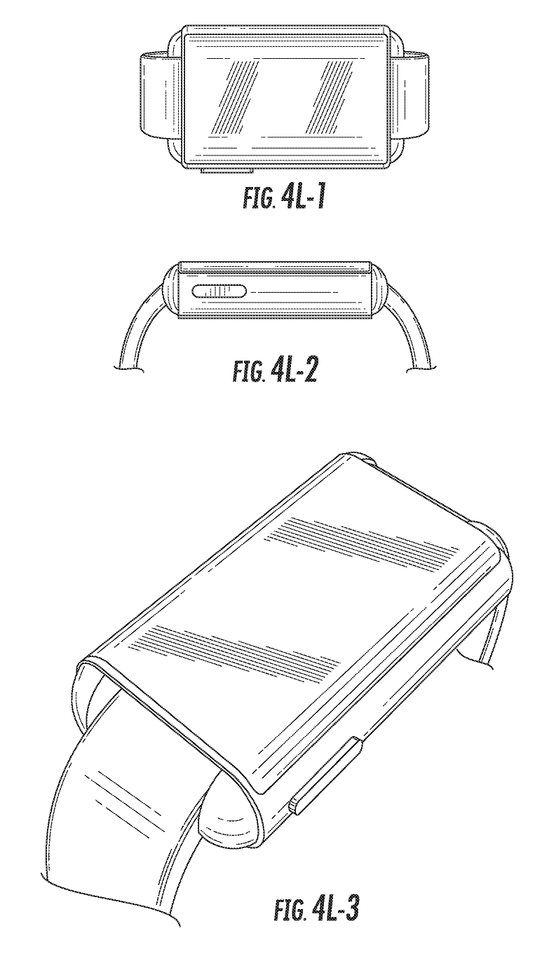

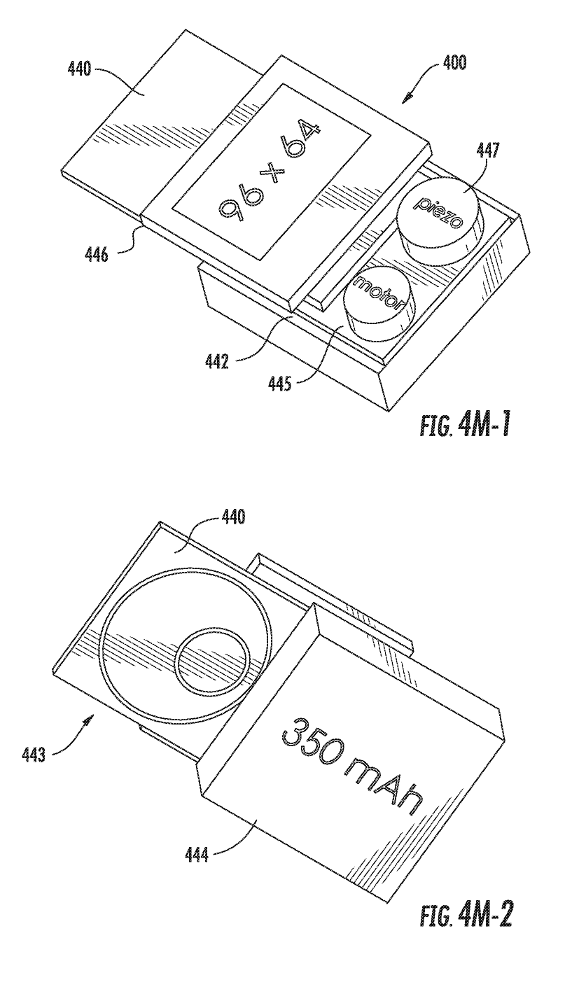

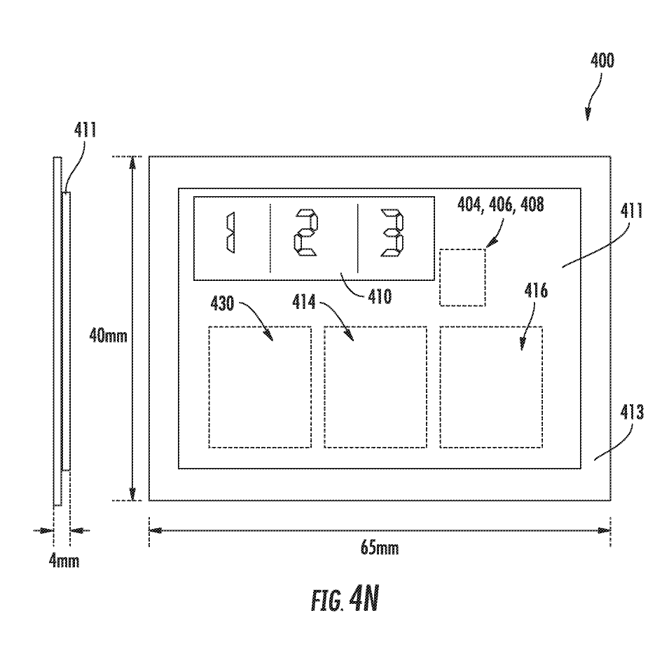

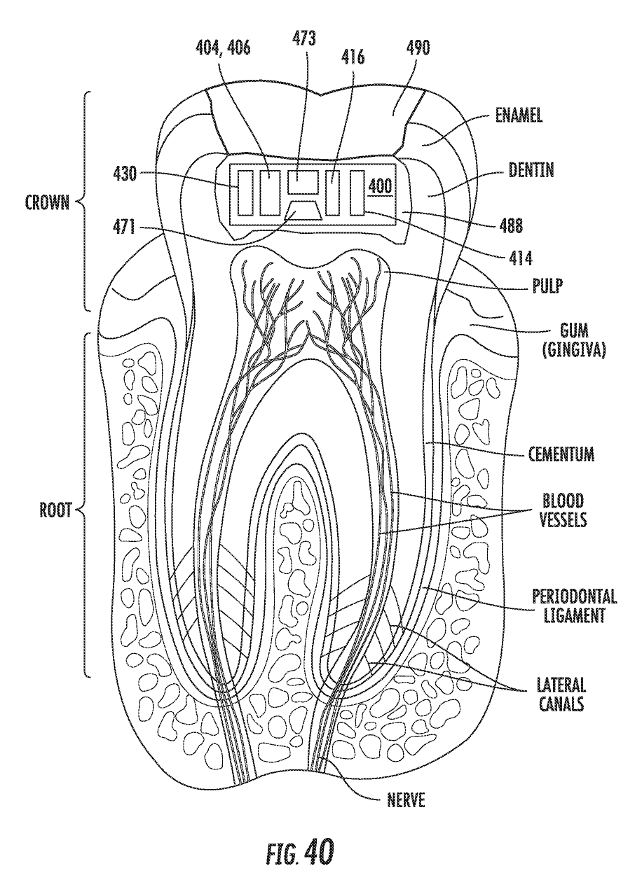

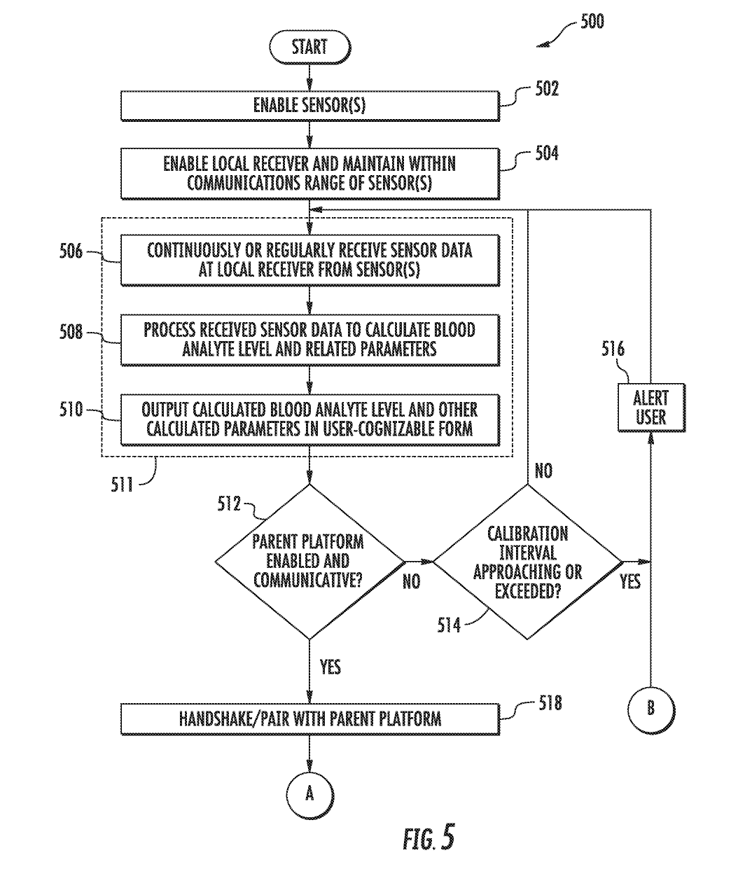

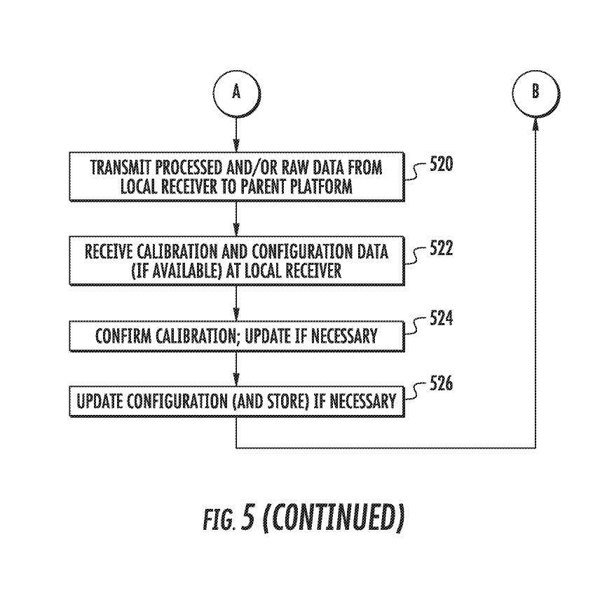

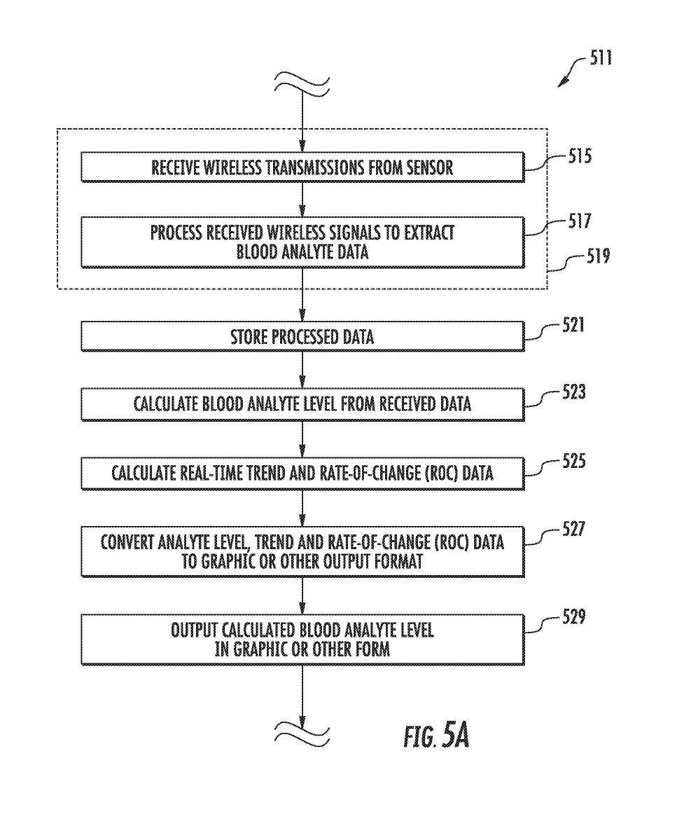

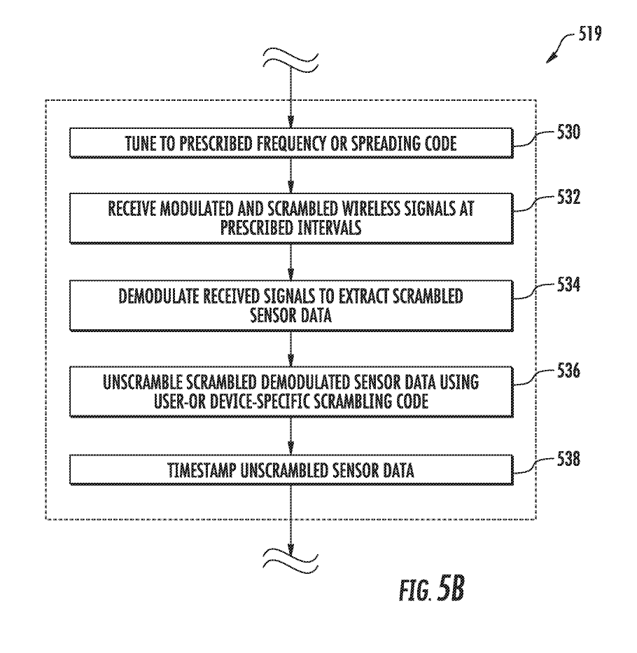

As shown in FIGS. 2-2C, the exemplary sensor apparatus 200 comprises a somewhat planar housing structure 202 with a sensing region 204 disposed on one side thereof (i.e., a top face 202a). As described in greater detail below with respect to FIGS. 4-5, the exemplary substantially planar shape of the housing 202 provides mechanical stability for the sensor apparatus 200 after implantation, thereby helping to preserve the orientation of the apparatus 200 and mitigate any tissue response induced by movement of the apparatus while implanted. Notwithstanding, the present disclosure contemplates sensor apparatus of shapes and/or sizes other than that of the exemplary apparatus 200.

The sensor apparatus of FIGS. 2-2C further includes a plurality of individual sensor elements 206 with their active surfaces disposed substantially within the sensing region 204 on the top face 202a of the apparatus housing. In the exemplary embodiment (i.e., an oxygen-based glucose sensor), the eight (8) sensing elements 206 are grouped into four pairs, one element of each pair an active or “primary” sensor with enzyme matrix, and the other a reference or “secondary” (oxygen) sensor. Exemplary implementations of the sensing elements and their supporting circuitry and components are described in, inter alia, U.S. Pat. No. 7,248,912, previously incorporated herein. It will be appreciated, however, that the type and operation of the sensor apparatus may vary; i.e., other types of sensor elements/sensor apparatus, configurations, and signal processing techniques thereof may be used consistent with the various aspects of the present disclosure, including, for example, signal processing techniques based on various combinations of signals from individual elements in the otherwise spatially-defined sensing elements pairs.

As discussed in greater detail below with respect to FIG. 5, the illustrated embodiment of FIGS. 2-2C includes a sensing region 204 which facilitates some degree of “interlock” of the surrounding tissue (and any subsequent tissue response generated by the host) so as to ensure direct and sustained contact between the sensing region 204 and the blood vessels of the surrounding tissue during the entire term of implantation (as well as advantageously maintaining contact between the sensing region 204 and the same tissue; i.e., without significant relative motion between the two).

The sensor apparatus 200 also includes in the exemplary embodiment a wireless radio frequency transmitter (or transceiver, depending if signals are intended to be received by the apparatus), not shown. As described in the aforementioned documents incorporated herein, the transmitter/transceiver may be configured to transmit modulated radio frequency signals to an external receiver/transceiver, such as a dedicated receiver device, or alternatively a properly equipped consumer electronic device such as a smartphone or tablet computer. Moreover, the sensor apparatus 200 may be configured to transmit signals to (whether in conjunction with the aforementioned external receiver, or in the alternative) an at least partly implanted or in vivo receiving device, such as an implanted pump or other medication or substance delivery system (e.g., an insulin pump or dispensing apparatus), embedded “logging” device, or other. It is also appreciated that other forms of wireless communication may be used for such applications, including for example inductive (electromagnetic induction) based systems, or even those based on capacitance or electric fields, or even optical (e.g., infrared) systems where a sufficiently clear path of transmission and reception exists, such as two devices in immediately adjacent disposition, or even ultrasonic systems where the two devices are sufficiently close and connected by sound-conductive media such as body tissues or fluids, or a purposely implanted component.

The sensor apparatus of FIGS. 2-2C also includes a plurality (three in this instance) of tabs or anchor apparatus 213 disposed substantially peripheral on the apparatus housing. These anchor apparatus provide the implanting surgeon with the opportunity to anchor the apparatus to the anatomy of the living subject, so as to frustrate translation and/or rotation of the sensor apparatus 200 within the subject immediately after implantation but before any tissue response (e.g., FBR) of the subject has a chance to immobilize (such as via interlock with the sensing region of the apparatus. See e.g., U.S. patent application Ser. No. 14/982,346 filed Dec. 29, 2015 and entitled “Implantable Sensor Apparatus and Methods” previously incorporated herein, for additional details and considerations regarding the aforementioned anchor apparatus 213 (which may include, for example features to receive sutures (dissolvable or otherwise), tissue ingrowth structures, and/or the like).

Moreover, another exemplary embodiment of the sensor apparatus 200 described herein may include either or both of: (i) multiple detector elements with respective “staggered” ranges/rates of detection operating in parallel, and/or (ii) multiple detector elements with respective “staggered” ranges/rates of detection that are selectively switched on/off in response to, e.g., the analyte concentration reaching a prescribed upper or lower threshold, as described in the foregoing Patent application Ser. No. 15/170,571.

The present disclosure further contemplates that such thresholds or bounds: (i) can be selected independent of one another; and/or (ii) can be set dynamically while the apparatus 300 is implanted. For example, in one scenario, operational detector elements are continuously or periodically monitored to confirm accuracy, and/or detect any degradation of performance (e.g., due to equipment degradation, progressive FBR affecting that detector element, etc.); when such degradation is detected, affecting say a lower limit of analyte concentration that can be detected, that particular detector element can have its lower threshold adjusted upward, such that handoff to another element capable of more accurately monitoring concentrations in that range.

It will be appreciated that the relatively smaller dimensions of the sensor apparatus (as compared to many conventional implant dimensions)—on the order of 40 mm in length (dimension “a” on FIGS. 2A-2C) by 25 mm in width (dimension “b” on FIGS. 2A-2C) by 10 mm in height (dimension “c” on FIGS. 2A-2C)—may reduce the extent of injury (e.g., reduced size of incision, reduced tissue disturbance/removal, etc.) and/or the surface area available for blood/tissue and sensor material interaction, which may in turn reduce intensity and duration of the host wound healing response. It is also envisaged that as circuit integration is increased, and component sizes (e.g., Lithium or other batteries) decrease, and further improvements are made, the sensor may increasingly be appreciably miniaturized, thereby further leveraging this factor.

It is also appreciated that some flexibility in component location exists; as such, the present disclosure further contemplates e.g., relocation of certain components within the implanted sensor device 200 such as those associated with signal processing, off-device (i.e., in a receiver module such as the local receiver described subsequently herein, or electronic apparatus external to the implanted sensor, such as a user's smartphone or tablet computer, or other implanted or external medical device) so as to further minimize interior sensor device volume/area requirements. For instance, in one such adaptation, electronic components such as antennas and/or circuit boards (e.g., PCBs) can be wholly or partly replaced with so-called “printable” electronics which reside on, e.g., interior components or surfaces of the sensor device 200 (or for that matter the local receiver 400 and/or output receivers 450, 452 described subsequently herein) such as by using the methods and apparatus described in U.S. Pat. No. 9,325,060 issued Apr. 26, 2016 and entitled “Methods and Apparatus for Conductive Element Deposition and Formation,” which is incorporated herein by reference in its entirety. Other types of space/area-reducing adaptations will be readily recognized by those of ordinary skill in the electronic arts when given the present disclosure.

### System Architecture—

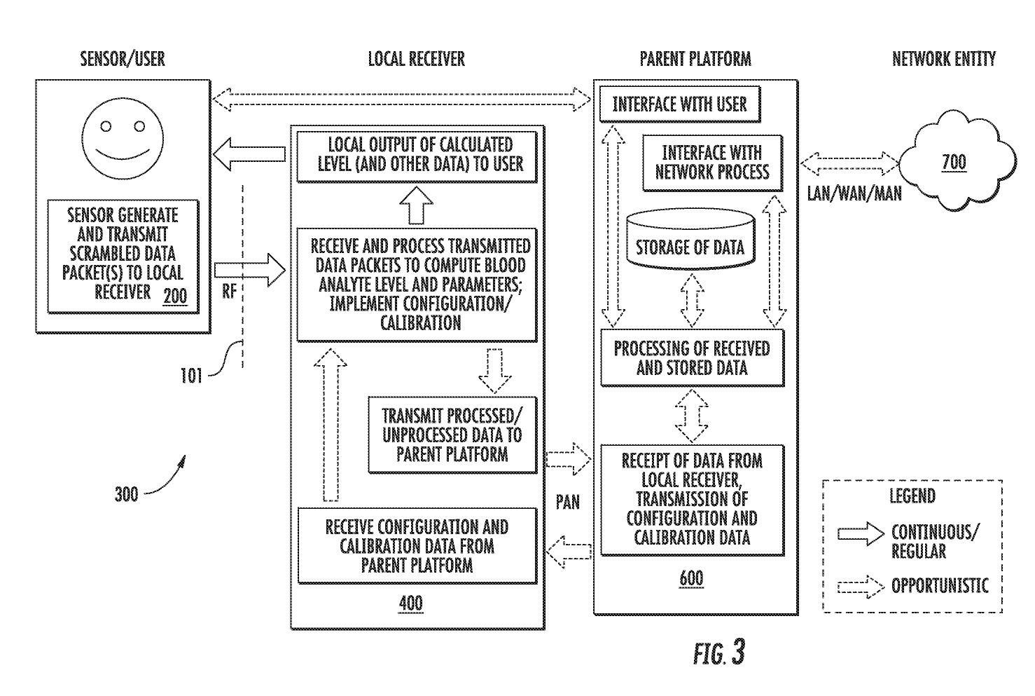

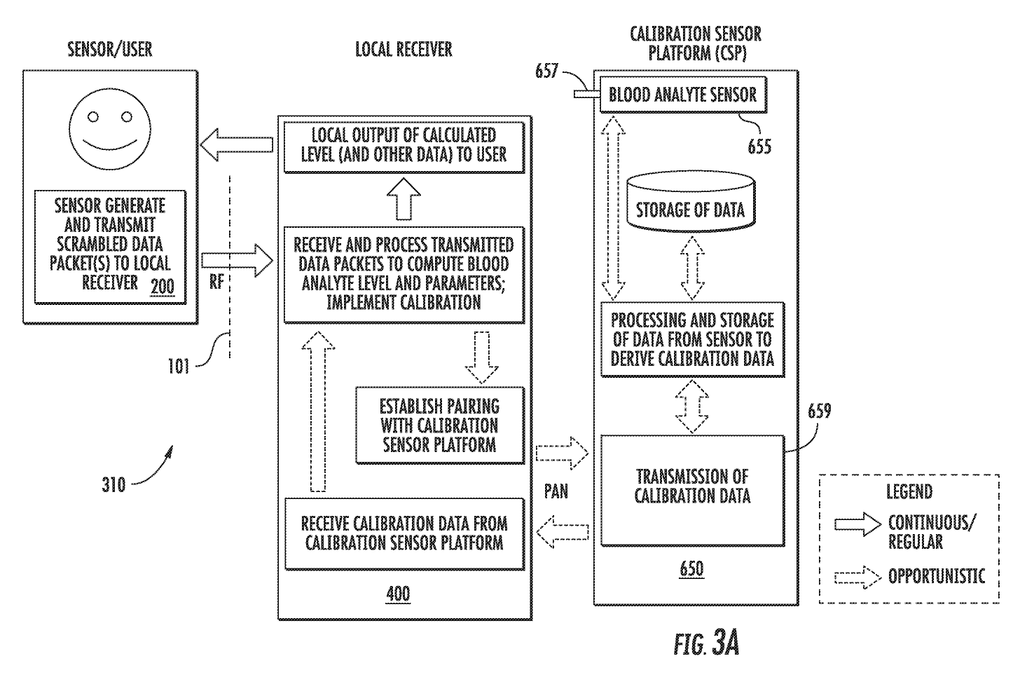

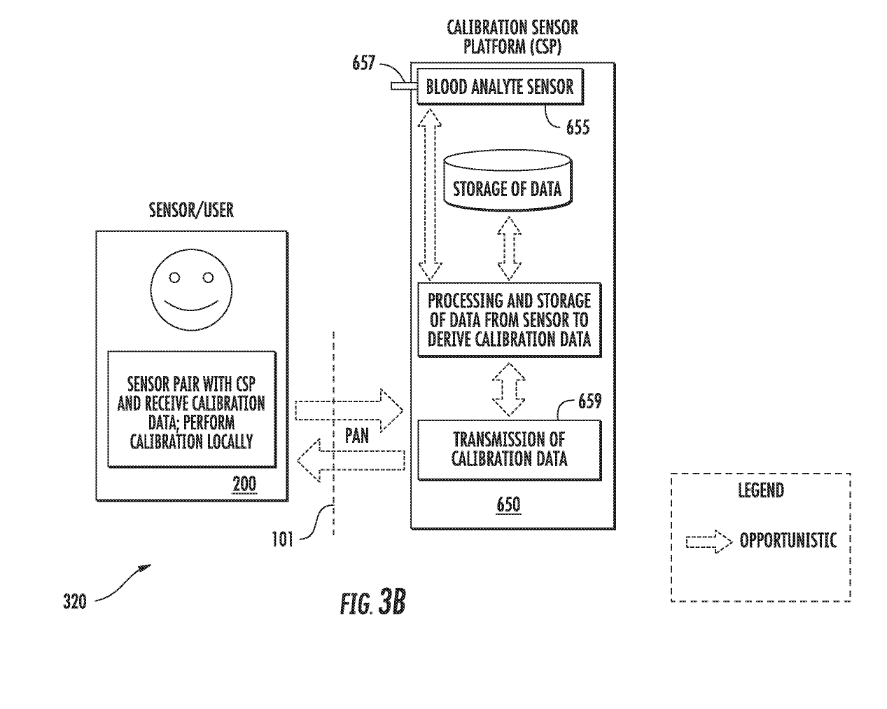

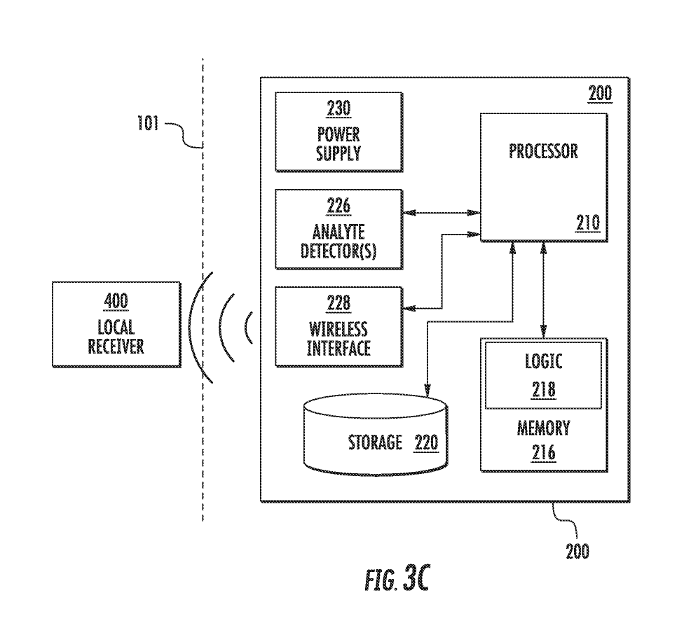

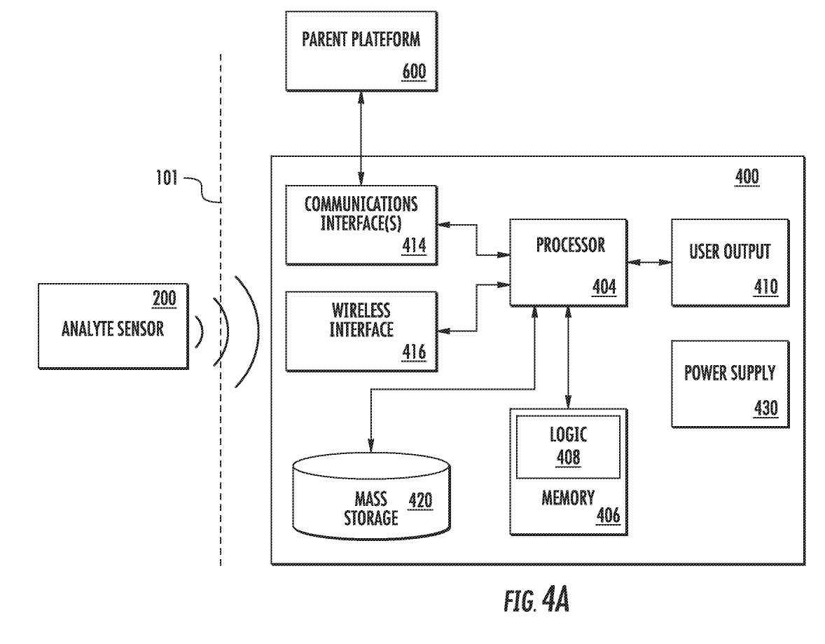

Referring now to FIG. 3, one embodiment of a system architecture for, inter alia, monitoring blood analyte levels within a user, according to the present disclosure is described in detail. As shown in FIG. 3, the architecture 300 comprises a sensor apparatus 200 (e.g., that of FIG. 2 discussed above, or yet other types of device) associated with a user, a local receiver 400, a parent platform 600, and a network entity 700. The sensor apparatus 200 in this embodiment communicates with the local receiver 400 via a wireless interface (described in detail below) through the user's tissue boundary 101. The local receiver 400 communicates (e.g., wirelessly) with the one or more parent platform(s) 600 via a PAN (e.g., Bluetooth or similar) RF interface, as discussed in greater detail below. The parent platform 600 may also, if desired, communicate with one or more network entities 700 via a LAN/WLAN, MAN, or other topology, such as for “cloud” data storage, analysis, convenience of access at other locations/synchronization with other user platforms, etc.

As indicated in FIG. 3, the communications between the sensor 200 and the local receiver 400 are generally “continuous” or regular in nature (i.e., happen according to a prescribed scheme and/or schedule), and hence are generally reliable in nature. In contrast, the communication between the local receiver 400 and the parent platform(s) is purposely “opportunistic” in nature; i.e., generally not according to any prescribed schedule or scheme, but rather when an opportunity presents itself. This is a significant advantage of the architecture 300 over the prior art; i.e., the ability for the sensor 200 and a reduced form-factor local receiver 400 to communicate regularly to enable reliable and effectively constant monitoring and user awareness of their blood analyte (e.g., glucose) level, without being “tethered” to larger, bulkier, and perhaps activity-limiting parent devices, including for extended periods of time. This functionality is enabled, in the exemplary embodiment, via the comparatively high degree of accuracy and calibration stability of the Assignee's oxygen-based blood analyte sensor described supra.

Specifically, in the illustrated architecture 300, the local receiver 400 acts as a reduced- or limited-functionality indicator and monitor for the user that reliably operates for comparatively extended periods of time without external input or calibration, thereby obviating the parent platform during those periods. As described later herein, the reduced form factor advantageously enables the user to further: (i) engage in activities which they could not otherwise engage in if “tethered” to the parent platform, and (ii) effortlessly keep the local receiver with them at all times, and obtain reliable blood analyte data and other useful information (e.g., trend, rate of change (ROC), and other sensor-data derived parameters), in a non-obtrusive (or even covert) manner.

When the opportunistic communication between the parent platform 600 and the local receiver does occur, the exemplary architecture 300 enables two-way data transfer, including: (i) transfer of stored data extracted from the sensor wireless transmissions to the local receiver, to the parent platform for archiving, analysis, transfer to a network entity, etc.; (ii) transfer of sensor-specific identification data and/or local receiver-specific data between the local receiver and the parent platform; (iii) transfer of external calibration data (e.g., derived from an independent test method such as a fingerstick or blood glucose monitor and input either automatically or manually to the parent platform) from the parent to the local receiver; and (iv) transfer of local receiver configuration or other data (e.g., software/firmware updates, user-prescribed receiver settings for alarms, warning/buffer values, indication formats or parameters, historical blood analyte levels for the user, results of analysis by the parent 600 or network entity 700 of such data, diagnoses, security or data scrambling/encryption codes or keys, etc.) from the parent 600 to the local receiver 400.

Referring now to FIG. 3A, another embodiment of a system architecture for, inter alia, monitoring blood analyte levels within a user, according to the present disclosure is described in detail. As shown in FIG. 3A, the architecture 310 comprises a sensor apparatus 200 associated with a user, a local receiver 400, and calibration sensor platform 650. As with the embodiment of FIG. 3, the sensor apparatus 200 in this embodiment communicates with the local receiver 400 via a wireless interface through the user's tissue boundary 101. The local receiver 400 communicates (e.g., wirelessly) with one or more calibration sensor platform(s) or CSPs 650 via a PAN (e.g., Bluetooth or similar) RF interface, as discussed in greater detail below, or via IR (e.g., IrDA-compliant), optical or other short-range communication modality. As described in greater detail below, the CSP 650 in the illustrated embodiment comprises a calibration data source for the local receiver 400, which may stand in the place of the more fully-functioned parent platform 600 for at least provision of calibration data.

As indicated in FIG. 3A, the communications between the sensor 200 and the local receiver 400 are again generally continuous or regular in nature while the communication between the local receiver 400 and the CSP 650 is purposely opportunistic in nature.

When the opportunistic communication between the CSP 650 and the local receiver does occur, the exemplary architecture 310 enables at least one-way data transfer, including transfer of external calibration data (e.g., derived from an independent test method such as the “fingerstick” or other form of blood analyte sensor 655 of the CSP 650 from the CSP to the local receiver 400. In an exemplary implementation, the CSP 650 comprises a “smart” fingerstick apparatus, including at least (i) sufficient onboard processing capability to generate calibration data useful with the local receiver 400 based on signals or data output from the blood sensor 655, and (ii) a data interface to enable transmission of the data to the local receiver 400. In one configuration, the sensor 655 includes a needle or lancet apparatus 657 which draws a sample of the user's blood for the sensor 655 to analyze. Electronic glucose “fingerstick” apparatus (including those with replaceable single-use lancets) and re-usable electronic components are well known in the relevant arts, and accordingly not described further herein. See e.g., U.S. Pat. No. 8,357,107 to Draudt, et al. issued Jan. 22, 2013 and incorporated herein by reference in its entirety, for one example of such technology. The sensor 655 analyzes the extracted blood obtained via the lancet 657 and (via the onboard processing) produces data indicative of a blood glucose level (or at least generates data from which such level may be derived), such data being provided to the communications interface 659 for transfer to the local receiver 400. The transmitted data are then utilized within the local receiver 400 for calibration of the data generated by the implanted sensor 200.

In one variant, the interface 659 comprises a Bluetooth-compliant interface, such that a corresponding Bluetooth interface of the local receiver can “pair” with the CSP 650 to effect transfer of the calibration data wirelessly. Hence, the user with implanted sensor 200 can simply use a fingerstick-based or other type of external calibration data source to periodically (e.g., once weekly) confirm the accuracy and/or update the calibration of the implanted sensor 200 via opportunistic communication between the local receiver 400 (e.g., small profile wristband, fob, etc.) when convenient for the user. Advantageously, many persons with diabetes possess such electronic fingerstick-based devices, and wireless communication capability is readily added thereto by the manufacturer at little additional cost.

In another variant, the communications interface comprises an IR or optical “LOS” interface such as one compliant with IrDA technology, such that the user need merely establish a line-of-sight path between the emitter of the CSP 650 and the receptor of the local receiver 400, akin to a television remote control. As yet another alternative, a near-field communication (NFC) antenna may be utilized to transfer data wirelessly between the apparatus 400, 650 when placed in close range (i.e., “swiped”). Yet other communication modalities will be recognized by those of ordinary skill given the present disclosure.

Referring now to FIG. 3B, yet another embodiment of a system architecture for, inter alia, monitoring blood analyte levels within a user, according to the present disclosure is described in detail. As shown in FIG. 3B, the architecture 320 comprises a sensor apparatus 200 associated with a user and the previously described calibration sensor platform 650. As with the embodiment of FIGS. 3 and 3A, the sensor apparatus 200 in this embodiment communicates with the local receiver 400 (not shown) via a wireless interface through the user's tissue boundary 101. However, the sensor apparatus 200 in this architecture is configured to communicate wirelessly with CSPs 650 via a PAN (e.g., Bluetooth or similar) or narrowband RF interface. As above, the CSP 650 in the illustrated embodiment comprises a calibration data source which provides calibration data, yet in this case the data are provided directly to the in vivo sensor 200, which is configured to utilize the data in generation of its own blood glucose concentration measurements or estimates. Such direct communication between the implanted sensor 200 and external CSP 650 might be useful or necessary when, for instance, the local receiver 400 is not present or available for communication with the sensor 200, such as when the user inadvertently leaves former at home on their way to work. So long as the user is in possession of the CSP 650, they can use the CSP to directly communicate data to the implanted sensor 200 (and use the CSP 650 user interface, not shown) to determine their then-current blood glucose level until, for example, they return home and place the local receiver 400 back in proximity and communication with the sensor apparatus as described elsewhere herein.

In one implementation, the sensor apparatus 200 is configured to store the generated blood glucose levels as well as the periodically received calibration data within onboard data storage, for later transfer to the local receiver 400. The calibration data can also be used by the sensor apparatus 200 before such transfer to calibrate, or confirm the accuracy of, the (internally) generated measurements of blood glucose, such that only calibrated/confirmed measurement data are stored (without having to also store or subsequently transfer the calibration data itself).

It will be appreciated that the architectures shown in FIGS. 3-3B are in no way exclusive of one another, and in fact may be used together (such as at different times and/or via different use cases). For instance, the architecture 320 of FIG. 3B can be used to supplement the architecture of FIG. 3 when for example the user does not have the local receiver 400 immediately in their possession or it is otherwise non-communicative with the sensor apparatus 200. Similarly, the architecture 310 of FIG. 3A can be used when the user's parent platform 600 (e.g., smartphone) is not available or communicative with the local receiver 400 for whatever reason. Myriad other permutations of use cases involving one or more of the various components 200, 400, 600, 650, 700 are envisaged by the present disclosure.

FIG. 3C is a functional block diagram illustrating an exemplary implantable sensor apparatus 200 and local receiver apparatus 400 according to one embodiment of the present disclosure. As shown, the sensor apparatus 200 includes a processor 210 (e.g., digital RISC, CISC, and/or DSP device), and/or a microcontroller (not shown), memory 216, software/firmware 218 operative to execute on the processor 210 and stored in e.g., a program memory portion of the processor 210 (not shown), or the memory 216, a mass storage device 220 (e.g., NAND or NOR flash, SSD, etc. to store collected raw or preprocessed data or other data of interest), one or more analyte detectors 226, a wireless interface 228 (e.g., narrowband, PAN such as Bluetooth, or other, described below), and a power supply 230 (e.g., a primary Lithium or rechargeable NiMH or Lithium ion battery). As can be appreciated by those of ordinary skill given the present disclosure, any number of different hardware/software/firmware architectures and component arrangements can be utilized for the sensor apparatus 200 of FIG. 3C, the foregoing being merely illustrative. For instance, a less-capable (processing, and/or data storage-wise) or “thinner” configuration may be used, or additional functionality not shown added (e.g., miniature accelerometer to, inter alia, enable detection of host movement and ambulatory state, orientation, etc.).

### Receiver Apparatus—

Referring now to FIGS. 4A-4O, various embodiments of the receiver apparatus 400 shown in FIGS. 3-3C herein are described in detail.

FIG. 4A is a functional block diagram showing one embodiment of the wireless receiver apparatus 400, in wireless communication with the analyte sensor 200 of FIG. 3C via the interposed tissue (boundary) 101. As noted previously, the present disclosure contemplates use of partially-implanted (e.g., transcutaneous) or even non-implanted analyte sensor devices, as well as the fully-implanted device (e.g., sensor apparatus 200 of FIG. 2).

As shown, the local receiver apparatus 400 includes a processor 404 (e.g., digital RISC, CISC, and/or DSP device), and/or a microcontroller (not shown), memory 406, software/firmware 408 operative to execute on the processor 404 and stored in e.g., a program memory portion of the processor 404 (not shown), or the memory 406, a mass storage device 420 (e.g., NAND or NOR flash, SSD, etc. to store received raw or preprocessed data, post-processed data, or other data of interest), a wireless interface 416 (e.g., narrowband or other, described below) for communication with the sensor apparatus 200, a communications (e.g., wireless) interface 414 for communication with the parent platform 600, and a power supply 430 (e.g., NiMH or Lithium ion battery, or other as described below). The apparatus 400 also includes one or more output device(s) 410 for communication of the desired data (e.g., glucose level, rate, trend, battery “low” alerts, etc.). As described in greater detail subsequently herein the output device(s) may include for example visual, audible, and/or tactile (e.g., haptic) modalities, which can be used alone or in concert depending on desired functionality and local receiver configuration.

As can be appreciated by those of ordinary skill given the present disclosure, any number of different hardware/software/firmware architectures and component arrangements can be utilized for the local receiver apparatus 400 of FIG. 4A, the foregoing being merely illustrative. For instance, a less-capable (processing, and/or data storage-wise) or “thinner” configuration may be used, or additional functionality not shown added (e.g., miniature accelerometer to, inter alia, enable detection of host movement and ambulatory state, orientation, etc.).

In one exemplary implementation, the protocol used to communicate between the in vivo device(s) (e.g., the sensor device 200 of FIG. 2) and the receiver 400 comprises an indigenous wireless protocol utilized by the O₂-based sensor (e.g., a 433 MHz wireless signal that is modulated with data according to a prescribed modulation type and data encoding format, or a standardized PAN interface such as Bluetooth; see discussion of FIGS. 5-5B infra). Hence, in one variant, the logic operative to run on the receiver (e.g., software “app”, firmware, etc.) 400 is configured to receive and process the O₂-based detector data e.g., for: (i) purposes of generation of an estimate of blood glucose level and output thereof to the user in a cognizable form; and (ii) communication to a parent platform 600 or cloud-based entity 700 (or another local receiver 400, such as in a peer-to-peer or P2P mode), as described in greater detail below with respect to FIGS. 5-5B.

FIG. 4B is a functional block diagram illustrating another exemplary embodiment of the local receiver apparatus of FIG. 3C, wherein a biocompatible (e.g., implanted) output receiver 450 is used in conjunction therewith. In this embodiment, the user has the output receiver 450 (e.g., a very small form-factor device capable of subcutaneous implantation or injection, or other implantation) disposed within their body, and the receiver 450 is configured to communicate with the local receiver 400 (or any other opportunistically present device configured to operate with the receiver 450). In one variant, the output receiver 450 includes or is part of an ancillary function related to the blood analyte level determined by the local receiver 400. For instance, an insulin delivery device may be fully or transcutaneously implanted in the user, and the local receiver output transmitter 422 can wirelessly communicate data such as blood glucose level, rate, trend, etc. to the output receiver 450 to enable pump operation, dosing, etc.

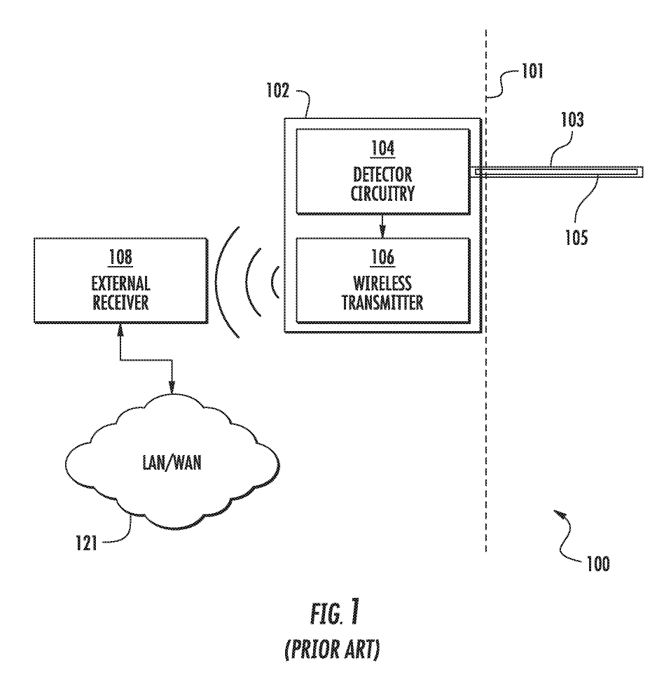

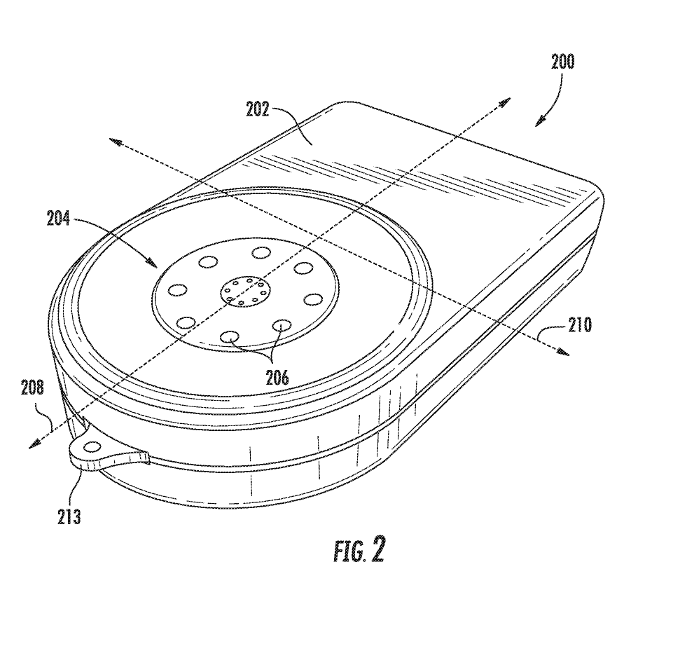

In another variant, the output receiver 450 is configured with a haptic or vibrational mode whereby data useful to the user can be encoded and communicated to the user directly through the user's anatomy. For instance, haptic “codes” of the type described subsequently herein with respect to FIGS. 4M-1 and 4M-2 can be generated through, e.g., incident electromagnetic energy capture from wireless signals produced by the local receiver transmitter 422, or other available “interrogator.” Hence, using this approach, the user can be interrogated much as one interrogates a passive RFID tag (i.e., using close range incident RF-frequency energy to excite electrical current flow within the antenna and supporting circuitry of the output receiver 450 to power the device to process and create the haptic output). The local receiver 400 merely acts as a pass-through for received and scrambled/encrypted data generated by the sensor 200, and need not unscramble or decrypt the sensor data signals, but rather pass them on in scrambled/encrypted fashion via the output transmitter 422 to the receiver 450, the latter capable of unscrambling/decrypting the signals to generate the desired (e.g., haptic) output. Hence, in this embodiment: (i) the user can utilize a “generic” local receiver apparatus 400 that has not been previously handshake or paired with the sensor 200 (i.e., the receiver 400 does not need to know the scrambling code), and (ii) the user's blood analyte data are never “in the clear” except when in vivo, and hence are more secure. So, for example, if the user forgets or loses their local receiver 400 (e.g., that of FIG. 4A) and is say, unable to obtain another one quickly, the biocompatible receiver 450 can act as a proxy or backup, wherein any generic device with capabilities of the local receiver 400 can enable the user to obtain a blood glucose reading. For instance, the present disclosure contemplates a kiosk, station, or other structure within e.g., a public place whereby the user can merely get in range of an interrogator antenna (e.g., 13.56 MHz ISO 14443 or 18000, NFC or similar), commence a “read” of the sensor apparatus 200 via another antenna of the same kiosk/station, and transfer the read (yet protected) data to the implanted receiver 450, thereby generating a haptic representation of blood glucose level or other parameters in a completely anonymous way.

In another configuration, the aforementioned “kiosk” or station functionality is integrated within the infotainment system of the user's car (not shown), such that the user can simply get in their car and cause readout of their blood analyte levels through e.g., an installed RF interrogator apparatus within the dashboard or other structure of the car. Moreover, it will be appreciated that the car infotainment system can in effect act like the local receiver 400 of FIG. 4A; i.e., include a wireless interface communicative with that of the sensor apparatus 200 such that the received data can be demodulated, unscrambled/decrypted if necessary, and both (i) displayed on the vehicle display device(s) (e.g., capacitive infotainment touch screen or TFT central display), read aloud via speech synthesis capability of the infotainment system, etc.; and (ii) be transmitted to a designated email address or social media account, network entity, server, process, etc. for later use, analysis, and/or distribution/synchronization with other user devices, via the vehicles indigenous wireless interface (e.g., LTE or Wi-Fi modem), such as when the user loses their wrist-worn or other “personal” local receiver 400.

In that the exemplary receiver 450 is passively powered, it arguably never needs explant unless it fails. Moreover, its form factor can much smaller than that of even the sensor apparatus, and can (and generally should) be implanted very superficially, such that the host experiences extremely little tissue trauma during the procedure.

It is appreciated that the receiver 450 of the embodiment of FIG. 4B can also be disposed external to the user's body (e.g., as a stick-on patch as described subsequently herein, such as with visual and/or haptic output modalities). See FIG. 4C.

FIG. 4D is a functional block diagram an exemplary embodiment of the output receiver of FIGS. 4B and 4C. In this embodiment, the receiver 450, 452 includes a wireless interface 464 configured to communicate with the output transmitter 422 of the local receiver 400, a microcontroller 454 (with processing logic 458 and memory 456), a controlled output device (e.g., haptic generator, display device, or insulin pump or other pharmacological or agent delivery system), and a power supply 480. As noted above, the power supply 480 can be combined or integrated with the wireless receiver 464 such that incident electromagnetic energy can be used to generate electrical power to operate the device 450, 452.

FIG. 4E is a functional block diagram illustrating yet a further exemplary embodiment of the local receiver apparatus 400 of FIG. 4A, wherein the local receiver apparatus is implanted within a host and communicates wirelessly with both a blood analyte (e.g., glucose) sensor and a parent platform. For instance, the apparatus 400 can be implanted subcutaneously as described above, and the user output device 420 can comprise a haptic apparatus that encodes signals perceptible by the user (i.e., under their skin). The wireless interface 416 can comprise e.g., the aforementioned 433 MHz narrowband system which is effective at propagating through human tissue, and hence the sensor 200 can communicate directly with the local receiver in vivo., or alternatively other types of interfaces with sufficient RF energy propagation through tissue such as a Bluetooth ISM-band (approximately 2.45 GHz) transceiver, or ZigBee PAN transceiver at approximately 915 MHz or 2.4 GHz.

Moreover, the implanted local receiver 400 can be configured such that the power supply 430 is passively activated (e.g., through incident RF energy, including that of the sensor 200, as described elsewhere herein), thereby obviating explants of the device (except for component failure).

The receiver apparatus 400 of FIG. 4E can also include a second wireless interface 414 for communication to the parent platform 600, such as a Bluetooth IC or similar. Notably, since the apparatus 400 is intended to be implanted superficially (e.g., subcutaneously, or in dental or prosthetic structures as described subsequently herein), the 2.4 GHz RF wavelength of the Bluetooth interface can propagate substantially unimpeded to the transceiver of the parent platform (contrast the “deep” implantation of the exemplary sensor apparatus 200).

FIGS. 4F-1 through 4L-3 are top, side, and perspective elevation views of various embodiments of a wearable local receiver apparatus according to the disclosure. As shown in the foregoing Figures, each of the embodiments comprises a generally small form-factor wrist-worn device, having a separate fabric or other material strap, or integral (e.g., molded yet flexible) retention mechanism. Each of the illustrated embodiments may have any combination of features appropriate to the particular application and/or user preferences, including without limitation one or more of: (i) waterproof or water resistant capability; (ii) shock and/or impact resistant capability; (iii) piezoelectric or other haptic output apparatus; (iv) LED or LCD display output; (v) acoustic output; (vi) function selection input device (e.g., button, tap sensor, or other), and (vii) external wireless interface (e.g., PAN, such as Bluetooth or Zigbee). Moreover, while not shown, it will be appreciated that the integral and even fabric-based embodiments of the apparatus may include one or more RF antenna components therein, such as to support the aforementioned exemplary 433 MHz and/or Bluetooth PAN interfaces of the receiver apparatus 400. Advantageously, the band comprises an appreciable surface area/volume within or on which such antenna components may be disposed, and furthermore allows for a wider band or range of frequencies to be supported than inclusion of one or more antenna elements solely within the body portion of the receiver apparatus 400.

FIGS. 4M-1 and 4M-2 are top and bottom perspective views, respectively, of another embodiment of the local receiver apparatus of the disclosure, configured to be deployable in a pendant or fob. In this embodiment, the receiver apparatus 400 comprises an antenna circuit board (e.g., PCB) 440, a main circuit board 442 with integrated circuit components such as the processor 404, memory 406, communications interface 414, and sensor wireless interface 416 (not shown), display device (e.g., LED or LCD-based device) 446, a microminiature DC vibration motor 445 (for e.g., haptic signaling to the wearer), a piezoelectric transducer element 447 (for e.g., acoustic signals, alarms, alerts, etc.), and battery 444 (e.g., a mAh-range Lithium ion or other battery). The antenna circuit board 440 includes in one embodiment the printed or deposited antenna conductive traces 443 as well as ground plane and other antenna components (not shown) necessary to support both the sensor interface 416 (e.g., at 433 MHz) and the secondary communications interface 414 (e.g., Bluetooth PAN). In the illustrated embodiment, overall dimensions of the apparatus 400 are on the order of 96 mm in length×64 mm in width×10 mm in height, although these dimensions and their relationship to one another are purely illustrative.

FIG. 4N is a front and side plan view of another embodiment of a user-wearable local receiver apparatus, configured as a flexible skin-adherent patch. In this configuration, the patch apparatus 400 comprises a flexible base substrate element 413 (such as e.g., a “flex” printed circuit board of the type known in the electronic arts, including a plurality of conductive traces formed thereon (not shown). The various integrated and discrete circuit components such as the processor 404, and memory 406, and the communications interface 414 and sensor wireless interface 416 and their associated antennas, are all disposed or formed onto or embedded into the substrate 413, as is a power supply device 430 (described in greater detail below) and display device 410. An upper layer 411 is formed onto (or laid atop and bonded to) the substrate element 413 so as to encase or enclose the various circuit elements therein (see side view of FIG. 4N). The illustrated implementation of the patch is on the order of 40 mm (width)×65 mm (length)×4 mm (thickness), although such dimensions are merely illustrative.

In the exemplary embodiment, a biocompatible and moisture-resistant adhesive (not shown) is also deposited onto the back surface of the substrate element 413, so as to permit temporary bonding of the patch apparatus 400 to the user's skin. Such adhesives are well known in the medical arts (suitable adhesives used for bandages, skin-attached appliances and the like), and accordingly are not described further herein.

Advantageously, the location of adhesion of the patch apparatus 400 is not significant for utilization of the apparatus; the user can place it literally anywhere it can adhere to (including under clothing, etc.) so as to be completely discrete.

In one implementation, the display device 410 comprises a substantially flat and flexible LED (e.g., graphene-based), AMOLED, or OTFT (organic thin-film transistor) display device which is configured to display desired information such as analyte concentration in the wearer's blood based on received signals transmitted from the implanted sensor and received via the sensor interface 416. Such OLED and OTFT-based elements are advantageously highly power efficient, and hence both conserve energy (thereby extending the lifetime of any static power supply used, such as a battery), and enable use of “dynamic” power supplies such as those described below which generally produce limited amounts of electrical power.

The patch apparatus 400 may be powered in one approach by a miniature, low-profile battery (e.g., Lithium-based device of the type well known in the art) with sufficient mAh capacity for the intended use lifetime (for instance, in the case of a disposable patch, the intended lifetime may be a few days or one week, and the battery is sized accordingly for that period).

In another exemplary configuration, the patch is powered by a flexible triboelectric or “static electricity”-based generator. See, e.g., Dhakar, Lokesh, et al, “Skin based flexible triboelectric nanogenerators with motion sensing capability”, 2015 28th IEEE International Conference on Micro Electro Mechanical Systems (MEMS) Jan. 18, 2015 (ISBN 978-1-4799-7955-4), incorporated herein by reference in its entirety, for one exemplary configuration of such tribolelectric generator apparatus useful with the present disclosure, although it will be appreciated that other types of device may be substituted. The aforementioned triboelectric nanogenerator (TENG) of Dhakar, et al uses an outermost layer of human skin (i.e., epidermis) as an active triboelectric layer for device operation, and generates an open circuit voltage of ˜90V with mild finger touch. The device uses PDMS nanopillar structures for the charging of two surfaces, and has been demonstrated as a wearable self-powered device which can be used as a motion and activity sensor as well as a power supply for electronic components such as those of the apparatus 400 of FIG. 4N. In one variant, the triboelectric generator is also coupled to an energy storage device (e.g., so-called “super-capacitor”, inductor, storage cell, or other) such that energy harvested by the triboelectric generator can be at least temporarily stored and used to support patch component functions even when no voltage is output from the generator. This storage configuration can also be readily applied to other “harvestable” dynamic energy sources described herein; e.g., radio frequency, solar radiation, etc.

In yet another variant, the patch apparatus 400 includes a passively powered (i.e., by incident electromagnetic energy) energy supply 430 which utilizes received RF energy from the sensor implant transmitter 200 (or another source, such as an external interrogator device) to power the components necessary to demodulate the incoming RF signal, unscramble it, extract the data from the demodulated and unscrambled signal, process the extracted data, and cause illumination of one or more light sources (e.g., ultra-low power LEDs) on the patch indicating the estimated analyte level. See, e.g., Takei, Ken et al; “Design for a 400-MHz Passive RFID Prototype System for Long Range Applications” Proceedings of ISAP2007, Niigata, Japan, January 2007, incorporated herein by reference in its entirety, which describes a passive radio frequency identification (RFID) system operating at 400 MHz using one or more of a distributed current antenna operating in a balanced circular polarized mode, a full wave rectifier fabricated from the balanced circuit independent from the RFID chip, and a feed forward circuit implanted with a carrier leak cancellation circuit (including a switch modulator, matching circuit, rectifier, and microprocessor) for, inter alia, extracting electrical power from the incident 400 MHz signal. See also “Wearable Flexible Lightweight Modular RFID Tag With Integrated Energy Harvester;” IEEE Transactions on Microwave Theory and Techniques, June 2016 (DOI: 10.1109/TMTT.2016.2573274), also incorporated herein by reference in its entirety, for alternative configurations. In this fashion, the “passive” patch apparatus 400 can extract electrical power to operate the display, processor, etc. from RF energy incident upon it, without need for a battery or other power source. In one such implementation, the antenna and matching circuitry of the patch power supply 430 (as contrasted to that associated with either the primary wireless receiver 416 or the secondary PAN transceiver 414) can be tuned to the frequency emitted by the sensor 200, such as 433 MHz, and make coincident use of the RF signal for energy. In another implementation, the primary wireless receiver 416 can be configured to perform both passive energy “harvesting” from the incident signal, as well as the aforementioned receipt and demodulation of the transmitted sensor data for ultimate unscrambling and extraction thereof (see FIG. 5).

It will also be appreciated that the implanted sensor 200 may also be configured with a rechargeable battery or storage cell and associated circuitry of the type known in the art, such that the battery or cell can be recharged in vivo, such as via an inductive charging approach, via energy harvested via movement of the host during their normal activities, or even via incident RF energy. In this fashion, any energy expended by the implanted device 200 to power the external patch apparatus 400 (or for other purposes, such as communication with other devices) can be offset through periodic recharging of the battery or cell.

In yet another variant, a photoelectric device (e.g., solar cell or array) can be used as the power supply 430 to the patch, whether alone or in combination with other supplies. It is appreciated that sufficient ambient light is needed to support operation of the patch 400 under purely solar power, which may not always be available (or, for instance, the patch 400 may be applied under the user's clothing for discretion). Hence, use of the foregoing solar cell(s) along with either (i) an energy storage device, or (ii) another, not-light dependent power supply is desirable in terms of user flexibility and reliability of the apparatus 400 for providing the desired monitoring and indication functions.

Moreover, regardless of power supply 430 configuration (i.e., triboelectric, battery, passive RF, motion-based, or solar), exemplary implementations of the patch apparatus 400 also include logic (e.g., rendered within the code executed on the processor 404, or even in hardware) which maintains various components of the apparatus (including the display and circuitry of the wireless interface 416) in a dormant state except when needed; i.e., when the user desires to observe the display, and/or when the wireless receiver of the patch must be powered on to receive modulated RF signals from the implanted sensor 200. For instance, in one variant, a portion of the pipeline of the processor apparatus 404 is shut down along with relevant portions of the display 410 and wireless interface 416, until a user-instigated event occurs (e.g., the user touches the patch, thereby generating a capacitive input) which wakes the dormant portion of the processor 404, which then executes instructions to wake the wireless interface 414, process the received wireless signals (i.e., demodulate and unscramble as described subsequently herein with respect to FIG. 5), and generate signals to drive the display device 410 to indicate a numeric value representative of the determined blood glucose value in, e.g. mg/dL or mmol/L. After expiration of a prescribed period of time (e.g., 5 sec.), the apparatus reverts to its “sleep” state so as to conserve power. See also the discussion of FIGS. 5-5B below regarding exemplary schemes for wireless transmission from the sensor apparatus 200, and reception by the local receiver so as to, inter alia, conserve on local receiver electrical power consumption.

In one implementation of the foregoing “patch” variants, the patches 400 are sold as a disposable commodity; e.g., a pack of twenty (20) which can be individually utilized by the user when a predecessor patch requires replacement due to loss, damage, or merely normal wear and tear. The patches can advantageously be manufactured as a low cost commodity (i.e., Bluetooth interface ICs, digital processors, memory, flexible PCBs, and other such components are currently highly commoditized and fungible in nature, and can be procured extremely inexpensively), and be designed for only a limited use lifetime, similar to prior art contact lens or other disposable biomedical products.

The patches can, as with other embodiments disclosed herein, also be made “self initializing” such that when initially activated, they handshake with the implanted sensor device 200 to ascertain the necessary scrambling code and other relevant data, such as transmission schedule for synchronization. In this manner, the user can merely enable a new (replacement) patch, and once synchronized with the sensor device 200, merely apply it to their skin in the desired location, and begin periodic monitoring using the new patch.

As yet other alternatives, the local receiver apparatus 400 may take the form of a badge or patch that can be temporarily or semi-permanently affixed to the user's clothing, hair accessory, eyeglass/sunglass frame, or yet other personal accessory.

In one implementation, a patch generally similar to that described supra with respect to FIG. 4N herein, yet is configured to be affixed to an extant device which the user commonly wears, such as their watch or smart watch. In one configuration, the patch adheres to the underside of the watch case, and provides the user with haptic output as to blood glucose level. For example, in one encoding scheme, the haptic apparatus encodes the actual value via a series of haptic codes (e.g., comprising: (i) a preamble (to alert the user that the data value is imminent), (ii) a “first code” for the third decimal place (e.g., hundreds), (iii) an “intermediate code” for the second decimal place (e.g., tens), and (iv) a “last code” for the first decimal place (e.g., ones). So, for example, the user might feel a comparatively “sharp” haptic impulse as a preamble to alert them to an impending blood glucose value, and a set of three discrete sets of impulses for the “hundreds”, “tens”, and “ones” places, each discrete set comprising a rapid succession of smaller impulses of equal intensity and duration, each set separated by e.g., a period of time so as to punctuate the sets for the user. Advantageously, in such scheme, the user only really need recognize the “hundreds” and “tens” impulses, as the maximum margin of error for failing to recognize the last (ones) impulses is small (i.e., 10 mg/dL). Moreover, the first (hundreds) impulses realistically will only encode 0 (for 0-99 mg/dL), 1 (for 100-199 mg/dl), and 2 (for 200-299 mg/dl); other values are largely non-physical. The haptic apparatus of the local receiver 400 can also be configured to only encode the first two decimal places (hundreds and tens) if desired as well, thereby shortening the time (and energy) needed to output the measured blood analyte level to the user (within the margin of error prescribed above).

Alternatively (or additionally), the haptic apparatus may simply encode a fuzzy logic or similar variable (e.g., one “buzz” or impulse for low, two in close sequence for moderate, three in sequence for high, and continuous for “acute/emergency” blood glucose levels). Other encoding schemes can be used for alerts as well, such as modulation of the intensity of the impulses (e.g., small impulse amplitude for a non-severe alert, higher amplitude for a greater urgency, etc.).

The foregoing encoding schemes (and yet others) can also be used in other form factors of local receiver 400 described herein if desired, such as the pendant/fob of FIGS. 4M-1 and 4M-2, or the wrist band(s) of FIGS. 4F-1 through 4L-3.

The applied “patch” or stick-on can also utilize display devices/formats similar to those of the apparatus 400 of FIG. N if desired, such as where the user adheres the device to say an existing prosthetic, piece of jewelry, etc. that is constantly carried on or with them, and which is readily visible to the user.

In another implementation, the blood analyte display/haptic functionality is incorporated into an extant electronic device, such as via a software/firmware upgrade and inclusion of an appropriate wireless interface and processing logic. For instance, in one such implementation, a smart watch with onboard processor, memory, display, WLAN interface (e.g., Wi-Fi), PAN wireless communication interface (e.g., Bluetooth or Zigbee compliant) and NFC (near field communication) interface can be used as the basis of the local receiver functionality. Specifically, in one approach, an “app” is downloaded onto the smart watch which is accessible by the user; the app controls operation of the Bluetooth or WLAN (receiver to parent) interface for opportunistic communications, as well as the NFC interface for communication with the sensor device. In the case of an implanted sensor device such as the device 200 of FIG. 2, the communication with the (nominally 13.56 MHz) passive or active NFC device of the smart watch occurs through tissue and over very short distances (at least at nominal power levels typically prescribed by such standards such as ISO 14443 or 18000; however, the user in such embodiments can merely place their arm with the watch thereon over the area of the abdomen where the sensor device 200 is implanted, thereby placing the NFC antenna of the watch within communications distance. In one such variant, the sensor 200 carries a secondary antenna capable of transmission of data at the prescribed NFC frequency (e.g., 13.56 MHz), and according to one protocol, the sensor 200 (i) receives a communication generated by the NFC IC of the watch (i.e., an “active” mode ping or handshake) to alert the sensor to the presence of the local receiver (watch), and (ii) in response, the sensor 200 wirelessly transmits the sensor data via the NFC antenna as opposed to the main (e.g., 433 MHz antenna), at sufficient power to be received by the external watch antenna without having to have it in very close proximity to the implanted sensor 200.

In a further variant, the apparatus comprises an “ear bud” or ear plug (or set thereof) which communicates with the user via audible output (and the implanted sensor and parent platform via its wireless interfaces 416, 414 respectively). In one implementation, the ear bud or plug is configured for wireless data communication with the implanted sensor and is battery powered, and the audible output comprises a synthesized voice readout of the numerical value of blood glucose level or other information of interest (see discussion of speech synthesis technology infra). In another implementation, the audible output comprises a series of discrete tones which encode the numeric value, and/or which are indicative of one or more alerts or action items for the host, similar to the haptic encoding scheme described supra.

Likewise, the foregoing functionality can be combined within or added to an extant hearing aid if present.

In yet a further variant, the apparatus comprises a ring or band worn on a user's finger, and which communicates with the user via haptic output (and the implanted sensor and parent platform via its wireless interfaces 416, 414 respectively). In one implementation, the ring is configured for wireless data communication with the implanted sensor 200 and is battery powered, and the haptic output (e.g., a small haptic oscillator embedded on the interior surface of the band contacting the user's skin) encodes the numerical value of blood glucose level or other information of interest (see discussion of haptic encoding schemes elsewhere herein). In one variant, the ring comprises a wedding band, which the user ostensibly wears at all times and hence is unlikely to be forgotten or lost due to its sentimental value. Other ring form factors are contemplated as well, such as engagement rings. university or alumni rings, etc., all which have a larger profile than the aforementioned wedding band (and hence more interior volume for components including a larger battery).

It is also appreciated that various of the devices 400 described herein can include a recharge capability; e.g., inductive charging by placing the device in proximity to a charging “plate” or other structure for a period of time, thereby enabling the magnetic inductance of the charger to induce electrical currents within the receiver charging circuit (not shown) to charge a rechargeable (e.g., Lithium-based) storage cell. While somewhat less desirable from the standpoint that the user must in effect monitor power level (as compared to a primary battery, which can last months or even years), such recharging capability can be used to achieve other desirable functions, such as an emergency “backup” capability (i.e., if the battery dies unexpectedly and no replacement is immediately available). An exemplary wireless charging circuit and device is described in U.S. Pat. No. 9,362,776 issued Jun. 7, 2016 and entitled “Wireless charging systems and methods”, incorporated herein by reference in its entirety, although other approaches may be used with equal success.

It is also appreciated that various of the embodiments of the receiver apparatus described herein may utilize (whether alone or in conjunction with other power sources) a thermo-electric power generation apparatus (not shown), for example one utilizing the Seebeck effect. As is well known, a thermoelectric device can be made using a thermocouple with two conducting paths with two different conductive materials (e.g., different metal alloys such as chrome and iron) or different semiconductors or a combination of a semiconductor and a metal alloy (e.g. p-doped silicon and copper). Between two open contact points a voltage V_{AB}, also referred to as the Seebeck voltage, is generated in the presence of a temperature gradient between the first and second end of the thermocouple. Such voltage can be used to, inter alia, power electrical devices (including the ICs and other components 404, 406, 410 of the local receiver), charge a storage cell, etc. See, e.g., U.S. Pat. No. 9,444,027 issued Sep. 13, 2016 and entitled “Thermoelectrical device and method for manufacturing same”, incorporated herein by reference in its entirety, for one exemplary configuration of, and method of manufacturing, a thermoelectric device useful with the present disclosure. The present disclosure appreciates that such local receiver apparatus, when in contact with various portions of the human body, may experience such a temperature gradient (e.g., due to ambient temperature differential, natural thermal gradients in the body, etc.), such that a Seebeck-based generator can be utilized, thereby further economizing on weight, space, and in some cases obviating any sizable energy storage device such as a battery.

### Implanted or Prosthetic Receivers—

In yet a further variant, the local receiver 400 of the architecture 300 of FIG. 3 apparatus comprises an implant or part of a user's extant prosthetic, which is used to receive signals transmitted from the implanted analyte sensor 200, and produce an output indicative of analyte level cognizable by the host. In one implementation (described with respect to FIG. 4O below), the implant comprises a dental implant with radio frequency receiver and an acoustic transducer, and is configured to receive RF transmissions from the implanted sensor at a prescribed frequency, demodulate and extract sensed analyte data, process the data, and generate a host-audible output relating to the analyte level (e.g., via transmission to the host's auditory system via the host's jawbone).

FIG. 4O is a side cross-sectional view of an exemplary implantable local receiver apparatus, configured as a dental implant. As shown, the local receiver apparatus 400 comprises an IC processor 404, memory 406, power supply (e.g., miniaturized battery) 430, Bluetooth or other parent platform interface 414, sensor device wireless interface 416, and an acoustic transducer 471 with supporting driver circuit 473. The receiver 400 is in the illustrated embodiment disposed within a central cavity region of the tooth (e.g., molar) and surrounded with an acoustically transmissive and biocompatible compound 488, the cavity sealed with a ceramic or even amalgam filling 490, although it will be appreciated that in other embodiments, the receiver is formed within a crown or other prosthetic (e.g., bridge) of the user which is then applied to the user in a semi-permanent fashion. For example, selectively actuated dental adhesive (e.g., that which can be degraded due to exposure to certain frequencies of UV or other types of electromagnetic radiation, or chemical substances), can be used in one implementation to allow for periodic battery replacement or change-out of the implant receiver 400, although it will be appreciated that other schemes may be utilized consistent with the present disclosure.

As can be appreciated, certain dental structures such as porcelain crowns, etc. have extremely limited space, and hence practical limitations on radio frequency interface selection are imposed due primarily to antenna dimension requirements. Hence, while not limited as such, the present disclosure primarily contemplates use of frequencies in the hundreds of MHz to GHz ranges (e.g., 400 MHz through 6 GHz) for both the wireless sensor interface 416 and the parent (e.g., PAN) wireless interface 414. Notably, many extant short range wireless communications standards such as Zigbee, Bluetooth, and RFID operate within such bands (whether licensed or unlicensed; e.g., 900 MHz, 2.4 GHz, 5.8 GHz), and hence are potential candidates for the wireless communications interfaces 414, 416, the primary or sensor interface 416 of the apparatus also limited by propagation of radio frequency energy (at a prescribed radiated power level) through the host's tissue when utilizing an implanted sensor such as the apparatus 200 of FIG. 2. As previously noted, frequencies on the order of 400-500 MHz tend to propagate well through human tissue, with decreasing propagation as frequency increases generally speaking. Hence, one embodiment of the implant apparatus 400 of FIG. 4O utilizes a 900 MHz ISM band unlicensed primary interface 416 for communication between the implant 400 and the implanted blood analyte sensor 200, and a 2.4 GHz Bluetooth PAN interface 414 for communication between the parent platform and the implant 400 (notably, only a small amount of tissue need be traversed, if any, for the RF energy of the secondary interface 416 to reach the parent platform wireless receiver, and vice versa). See e.g., United States Patent Application Pub. No 20160134980 published May 12, 2016 and entitled “METHODS AND APPARATUS FOR PROCESSING AUDIO SIGNALS” (describing various configurations for wireless-enabled dental implants and prosthetics), as well as Ishihata, H. et al, “A radio frequency identification implanted in a tooth can communicate with the outside world”, IEEE Transactions Inf. Technol. Biomed. 2007 November; 11(6):683-5 (describing a radio frequency identification (RFID) transponder covering the 13.56 MHz band adapted to minimize its volume for placement in the pulp chamber of an endodontically treated human tooth and capable of communication with a reader), each of the foregoing incorporated herein by reference in its entirety.

In one implementation of the apparatus of FIG. 4O, the (scrambled) wireless data signals are received by the wireless interface 416 as described elsewhere herein, and demodulated, unscrambled, and the data extracted. The extracted data are then processed by the processor 404 and onboard software (not shown) to generate an estimate of blood analyte level. This estimate comprises a binary form of a numeric value in the units converted (e.g., mg/dL or mmol/L), and this binary value is then converted to a series of tones by the digital-to-analog conversion apparatus and driver circuit 473, coupled electrically to a micro-miniature transducer element 471. See, e.g., the exemplary transducer device described in U.S. Pat. No. 8,270,661 to Sorensen, et al. issued Sep. 18, 2012 and entitled “High efficient miniature electro-acoustic transducer with reduced dimensions”, incorporated herein by reference in its entirety, for one exemplary configuration of a miniature transducer suitable for use with the apparatus 400 of FIG. 4O, although other devices and configurations may be used as well. It is appreciated that while human hearing nominally falls within the range of 20 Hz to 20 KHz, the apparatus described herein may utilize a significantly smaller band (and hence dynamic range of the transducer and its driver circuitry), thereby enabling further miniaturization, such as by obviating the need for the generally larger structures required to create low frequencies below e.g., 100 Hz. Stated simply, the transducer and driver circuitry can be made smaller and less expensively if it need only support a narrow dynamic range.

In another implementation, the processor 404 of the dental implant 400 of FIG. 4O includes a speech synthesis algorithm operative to execute thereon (e.g., stored in program memory of the processor 404, or in the storage device 406) so as to generate a language-based representation of the analyte level binary data. In the exemplary configuration, the speech library of the algorithm is limited to only numeric values and certain keywords for alerts (e.g., “low battery”, “High Glucose Warning” and the like), so as to reduce code size and storage requirements, although more expanded libraries can be used as well. Myriad approaches to speech synthesis from e.g., text or binary data are known in the art (see e.g., U.S. Pat. No. 9,002,711 issued Apr. 7, 2015 and entitled “Speech synthesis apparatus and method”, incorporated herein by reference in its entirety, as one exemplar of such technology), and may be used consistent with the present disclosure.

In operation, the implant apparatus 400 of FIG. 4O generates acoustic-range output at a prescribed volume level (which is comparatively significantly lower than normal speech in terms of db, since the transmission of the acoustic vibrations are through the tooth dentin and other physiologic structures to include the jaw bone), and the user can hear the acoustic output directly via their inner ear structure; transmission through the tympanic membrane is obviated, and hence the user is the only one who can hear the output.

### Methods—

Referring now to FIGS. 5-5B, exemplary embodiments of the methods of operating the local receiver apparatus (and analyte sensing system generally) are described in detail.

FIG. 5 is a logical flow diagram illustrating one exemplary embodiment of a method 500 of operating a local receiving device for blood analyte measurement according to the present disclosure.

As shown in the Figure, the method 500 begins with the user or clinician enabling the sensor (e.g., implanted device 200 of FIG. 2, or other) per step 502. In the case of the implantable sensor of FIG. 2, the sensor is enabled, implanted in the host (such as via the procedures described in U.S. patent application Ser. No. 14/982,346 filed Dec. 29, 2015 previously incorporated herein), and tested as part of step 502.

Next, the local receiver apparatus 400 (e.g., any of those of FIGS. 4A-4N herein) is enabled, and maintained within communications range of the sensor apparatus, per step 504. As noted supra, the exemplary embodiment of the sensor apparatus uses a 433 MHz narrowband RF transmitter (such frequency having good signal transmission characteristics through human tissue), and hence has a communications range, dependent on transmission power, of at least several feet. Hence, in one implementation of the method step 504, the host/user merely needs to keep the local receiver 400 within arm's reach, or somewhere on their body personally.

Per step 506, the enabled sensor 200 communicates data wirelessly to the local receiver 400, such as on a periodic, event-driven, or other basis. Note that the transmission and reception frequencies or schedule need not necessarily coincide completely. For instance, the transmitter of the sensor apparatus 200 may transmit according to a prescribed periodicity or frequency, while the local receiver 400 may utilize a less frequent sampling of the transmissions.

In one such implementation, the wireless signals are transmitted from the sensor device e.g., only at prescribed times or prescribed intervals, and the apparatus is configured to synchronize the schedule, and enable the components of the wireless receiver apparatus to receive the wireless signals only during the prescribed times or at the prescribed intervals, and otherwise maintain at least a portion of the wireless receiver apparatus in a dormant or sleep state so as to conserve electrical power. For example, the processor 404 may only “wake up” the receiver 416 and other components for reception of the prescribed events, and then return them to a sleep state thereafter.

In a further variant, the wireless signals are transmitted from the sensor device 200 only at prescribed times, and the wireless receiver apparatus is configured to receive the wireless signals during a number n of prescribed times, the number n being less than a total number of transmissions of wireless signals. The local receiver logic can further be configured to dynamically vary the number n based at least on one or more operational parameters, such as e.g., a remaining level of power in the electrical power source (e.g., battery level, as determined by a known voltage versus capacity profile), a time period from when a last prior calibration was applied to the data relating to levels of the blood analyte (e.g., when was the last calibration data input received from the parent platform), the determined blood analyte level (i.e., sampling may vary and become more frequent as the detected blood glucose level approaches an alert or boundary level), and/or detection of an ambulatory or non-ambulatory state of the user (e.g., the sampling frequency can be reduced when the user is asleep, or otherwise in a state where the rate of change of blood glucose level is expected to be substantially stable).

Per step 508 of the method 500, the received sensor data are processed to calculate blood analyte level, and any related parameters or data derived therefrom. Such processing may occur when the data are received, or collectively in one or more aggregations or batches of data (e.g., sensor data collected or received over a prescribed time period).

Per step 510, the calculated blood analyte level (e.g., glucose concentration in e.g., mg/dL or mmol/L) is output to the user in a cognizable form, such as visually, via haptic apparatus, audibly, and/or yet other means, as described elsewhere herein. Similarly, other information (such as trend of the blood glucose level, rate of change, and/or alerts) may be output from the local receiver 400 via the same or different cognizable medium. For instance, in one embodiment, the blood glucose level is displayed on a display device of the local receiver as a sequence of numbers (e.g., “123”), while alerts or warnings are output as audible tones or “chirps,” and/or haptic pulses to the user via the local receiver's contact with their skin. Numerous permutations of the foregoing will be appreciated by those of ordinary skill given the present disclosure.

Per step 512, the method 500 further determines whether the parent platform 600 (e.g., the user's more fully-functioned tablet, smartphone, etc.) is “communicative” with the local receiver 400. This determination may be made actively or passively, periodically or based on an event, and directly or indirectly. Specifically, the method step 512 contemplates various different approaches to determination of whether communications can (or should) be established with the parent platform 600, including without limitation: (i) evaluating a signal strength (such as a Bluetooth RSSI or other metric) of a beacon or other signal transmitted by a wireless interface of the parent platform; (ii) issuing a probe or other communication signal or request, and evaluating any response thereto (or lack thereof); (iii) receiving one or more communications (e.g., messages) from an application layer process of the parent platform, indicating e.g., either current availability/readiness for data communication (one-way or two-way), a “back-off” period after which the local receiver can/should attempt communication again, or other information relating to one- or two-way data transfer between the local receiver and parent platform (such as the presence of a software/firmware update for the local receiver). The foregoing step of the exemplary embodiment is considered “opportunistic” in the sense that such communication between the local receiver and the parent platform is required only very infrequently (e.g., once a week, or even less frequently), owing in large part to the excellent stability and reliability of the exemplary implanted blood analyte sensor 200 over time. This underscores one salient advantage of the architecture of the present disclosure; i.e., the ability of the user to divorce themselves (and the local receiver) from the parent platform for extended periods of time, and in effect only enable communication between the parent and local receiver when convenient (e.g., once a week, or less).

Returning to FIG. 5, when communications are enabled per step 512, the local receiver and parent platform handshake (e.g., pair according to a Bluetooth pairing protocol, with the local receiver as the slave, and the parent as the master). In one implementation, the master/slave architecture inherent in the Bluetooth topology is advantageously leveraged to enable multiple simultaneous pairings between a single parent platform and two or more local receivers (e.g., associated with two or more respective individuals), such that “group updates” can be performed substantially simultaneously. For instance, in one use case, two family members with implanted blood analyte sensors 200 (e.g., husband/wife, mother/child, etc.) can each enable pairing with a common parent platform, such as the mother's smartphone in the prior example, and updates/configuration changes can be inserted, and calibration performed as needed, for both devices, thereby obviating each user having to associate with a separate parent platform. This type of approach is also useful in, inter alia, instances where one user is a juvenile, and may not fully comprehend or appreciate the ramifications of certain outputs from the local receiver, or how to properly configure or calibrate their own local receiver/sensor system.

To that end, the present disclosure also contemplates varying types of local receiver apparatus 400; e.g., for different age groups, with features and functionality particularly adapted for that age group. For instance, a “senior” device might include larger numerals for easy readability, alerts with mandatory acknowledgements to confirm that an action was taken (e.g., ingestion of a certain food or medication), etc. Similarly, a “juvenile” version might include the aforementioned parental control functions, be limited in terms of settings or other features that can be altered by the juvenile, a GPS-based “child locator” function, etc.

Hence, one implementation of the method (and underlying system architecture) contemplates “parental controls” of sorts, such that the parent platform of the controlling user (e.g., mother) is configured to control one or more functions of the controlled party's (e.g., child's) local receiver and its interaction with the parent platform (e.g., mother's smartphone and installed application layer software), so that (i) any significant events such as blood glucose transients are not missed and are properly “alarmed,” and (ii) proper calibration is conducted (if needed), the foregoing controlled from the parent platform application software user interface (UI) which recognizes both local receivers, and maintains configuration and calibration data for both.

Per step 522 of the method 500, the local receiver(s) receive the configuration and/or calibration data as applicable from the parent platform, and per step 524, utilize the received data to confirm calibration of the sensor e.g., through comparison of “fingerstick” or blood glucose monitor (BGM) values entered by the user via the parent platform software UI. In one implementation, when calibration is required (e.g., the received or external calibration data varies from the corresponding value calculated by the local receiver based on the sensor data more than a prescribed amount), the user is presented with a “OK to update calibration?” or similar message or indication, requiring the user to affirmatively confirm insertion of the calibration data. In other implementations, such updates can be: (i) automatically inserted; (ii) inserted after a sufficient number of independent external data points (and sensor-based calculated values) are available for averaging or other statistical or algorithmic analysis; or (iii) inserted only to a permissible level of change (e.g., not to exceed 5% variation from the extant sensor-based value). Myriad other permutations or variations on the foregoing will be appreciated by those of skill in the art given the present disclosure.

Per step 526, the configuration of the local receiver (e.g., the alarm setting values, alert logic or hierarchy such as “haptic then visual then audible”, etc.) is also updated as needed.

Alternatively, if the parent platform is not “communicative” (e.g., outside range, busy, preempted, etc.) per step 512 of the method, the calibration status of the local receiver 400 is determined by, for instance, the onboard logic of the local receiver per step 514. For example, in one implementation, the status check of step 514 comprises determining the relationship of a time since last calibration/update to a prescribed value stored in memory as part of the initial configuration of the device (e.g., N days). So, if the local receiver 400 has for instance not received any external calibration data for six (6) days, and the user has pre-configured the “alert” level for calibration to be six days, the local receiver will generate a visual, haptic, and/or audible alert for the user per step 516, in effect alerting the user for the need to pair the local receiver to the parent platform (or otherwise confirm the accuracy of the calculated value, such as via fingerstick test and direct comparison by the user). If the preconfigured threshold or alert level has not been exceeded, the method 500 returns to step 506, where periodic receipt and processing of sensor data is continued.

FIG. 5A is a logical flow diagram illustrating one exemplary implementation of the sensor data processing and output methodology 511 according to the method 500 of FIG. 5. As shown, the method 511 in one embodiment includes first receiving the wireless data transmissions from the (implanted) sensor 200 per step 515. Next, per step 517, the received wireless signals are processed (see the exemplary method of FIG. 5B, discussed below), and the processed data stored (step 521). Blood analyte level is calculated per step 523, and also other parameters of interest if any (such as real-time trend and/or rate of change) are calculated per step 525. The calculated values from steps 523, 525 are then converted per step 527 to a prescribed output format (e.g., a graphic rendering of a numeric value, a graphic display of a trend arrow, a sequence of haptic vibrations, etc.) consistent with the selected/configured output modality. The converted values or indications are then output to the user in the appropriate modality/modalities per step 529.

FIG. 5B is a logical flow diagram illustrating one exemplary implementation of the sensor data receipt and demodulation/unscrambling methodology 519 according to the method 500 of FIG. 5A. In the illustrated embodiment of the method 519, the wireless receiver 416 of the local receiver apparatus 400 tunes to the appropriate center frequency of the sensor transmitter (e.g., 433 MHz) if required per step 530. Alternatively, in the case of a spread spectrum transmission/reception scheme, the pseudo-noise (pn) spreading code (for e.g., CDMA or other DSSS systems) is used by the local receiver to extract the desired signals, or a hopping sequence shared by transmitter and receiver is accessed by the receiver to extract the signals. Likewise, time-frequency resources are accessed in an OFDM-based system for signal extraction.

In the exemplary implementation, regardless of spectral access technique, scrambled sensor data are modulated onto the carrier(s) by the transmitter of the sensor apparatus 200 to encode “raw” sensor data, for use by the local receiver at step 532. The data are scrambled before modulation onto the carrier(s) by a scrambling algorithm operative to run on the sensor apparatus 200 in order to maintain some degree of user/data privacy and avoid surreptitious interception and use, although it will be appreciated that other approaches may be used (e.g., the data may be unscrambled but encrypted according to an AES/DES algorithm or public/private key pair scheme, cryptographically hashed using a one-way hash algorithm, etc.). In one implementation, the scrambling is conducted according to a prescribed sequence; i.e., based on data unique to the particular local receiver (e.g., MAC-64 or EUI 64 MAC address). The local receiver 400 knows only its own unique data, and hence can only unscramble wireless transmissions from its own “host” sensor, thereby avoiding situations where one user's local receiver receives and unscrambles the data transmitted from another user's implanted sensor 200.

In another variant, the scrambling is conducted based on a concatenation or combination of (i) the unique data of the local receiver 400, and (ii) unique data of the sensor 200, such that the local receiver 400 must know both sets of unique data before it can unscramble the received signals (such as data exchange via an initial “pairing” or handshake of the user's particular local receiver and their implanted device, e.g., at time of purchase of the local receiver or implantation of the sensor 200).

Next, per step 534, the received modulated and scrambled signals are demodulated at the local receiver to extract the scrambled data signal. For example, the signals may have been modulated onto the carrier(s) by the transmitter using any number of different schemes, such as FSK, QPSK, DPSK, ASK, GMSK, QAM, etc., and accordingly are demodulated by the receiver according to the same scheme.

The demodulated and scrambled signals are then unscrambled per step 536 (as described above), such as using a device-specific or unique scrambling code. After unscrambling, the “raw” transmitted sensor data packet is then timestamped per step 538 so as to preserve its temporal information (including ordering of the data), and stored within the local receiver's memory device (e.g., flash memory) for subsequent use per step 521 of FIG. 5A.

It will be recognized that while certain embodiments of the present disclosure are described in terms of a specific sequence of steps of a method, these descriptions are only illustrative of the broader methods described herein, and may be modified as required by the particular application. Certain steps may be rendered unnecessary or optional under certain circumstances. Additionally, certain steps or functionality may be added to the disclosed embodiments, or the order of performance of two or more steps permuted. All such variations are considered to be encompassed within the disclosure and claimed herein.

While the above detailed description has shown, described, and pointed out novel features as applied to various embodiments, it will be understood that various omissions, substitutions, and changes in the form and details of the device or process illustrated may be made by those skilled in the art without departing from principles described herein. The foregoing description is of the best mode presently contemplated. This description is in no way meant to be limiting, but rather should be taken as illustrative of the general principles described herein. The scope of the disclosure should be determined with reference to the claims.

1.-53. (canceled)

54. An implantable blood glucose sensor apparatus, comprising:
at least one detector element configured to generate signals related to a blood glucose level when the implantable blood glucose sensor apparatus is implanted within tissues of a subject;
wireless interface apparatus configured for wireless data communication with one or more computerized devices, the one or more computerized devices comprising at least a receiver apparatus;
data processor apparatus in data communication with the at least one detector element;
data storage apparatus in data communication with the data processor apparatus, the data storage apparatus having at least one computer program stored thereon, the at least one computer program comprising a plurality of instructions, the plurality of instructions configured to, when executed by the data processor apparatus, cause the implantable blood glucose sensor apparatus to:
operate the implantable blood glucose sensor apparatus for a period of time autonomous of the receiver apparatus, the autonomous operation comprising at least:
collection of the signals generated by the at least one detector apparatus over the period of time;
processing of at least a portion of the signals using at least the data processor apparatus to produce processed signal data;
utilization of the processed signal data to generate data indicative of a plurality of blood glucose level measurements associated with the period of time; and
storage of the data indicative of the blood glucose level measurement over the period of time in the data storage apparatus.

55. The implantable blood glucose sensor apparatus of claim 54, wherein:
the plurality of instructions are further configured to, when executed by the data processor apparatus, cause the implantable blood glucose sensor apparatus to prior to the autonomous operation, receive calibration data transmitted from the at least one of the one or more computerized devices via the wireless interface apparatus; and
the autonomous operation further comprises utilization of the received calibration data in generation of the data indicative of the plurality of blood glucose level measurements associated with the period of time.

56. The implantable blood glucose sensor apparatus of claim 54, wherein the plurality of instructions are further configured to, when executed by the data processor apparatus, cause the implantable blood glucose sensor apparatus to:
after the period of time, establish wireless data communication with the receiver apparatus via at least the wireless interface apparatus; and
based at least on part on the establishment of the wireless data communication, wirelessly transmit at least a portion the data indicative of the plurality of blood glucose level measurements associated with the period of time to the receiver apparatus.

57. The implantable blood glucose sensor apparatus of claim 54, wherein the autonomous operation further comprises:
utilization of the data indicative of the plurality of blood glucose level measurements associated with the period of time to generate one or more of trend data or rate of change (ROC) data; and
storage of the one or more of the trend data or the ROC data in the data storage apparatus.

58. The implantable blood glucose sensor apparatus of claim 57, wherein the plurality of instructions are further configured to, when executed by the data processor apparatus, cause the implantable blood glucose sensor apparatus to:
after the period of time, establish wireless data communication with the receiver apparatus; and
wirelessly transmit the generated one or more of the trend data or the ROC data to the receiver apparatus, the receiver apparatus configured to convert the generated one or more of the trend data or the ROC data to a prescribed output format for user notification.

59. The implantable blood glucose sensor apparatus of claim 54, wherein:
the plurality of instructions are further configured to, when executed by the data processor apparatus, cause the implantable blood glucose sensor apparatus to, prior to the autonomous operation, identify that the receiver apparatus is non-communicative with the implantable blood glucose sensor apparatus; and
the operation the implantable blood glucose sensor apparatus for the period of time autonomous of the receiver apparatus is based at least in part on the identification that the receiver apparatus is non-communicative with the implantable blood glucose sensor apparatus.

60. The implantable blood glucose sensor apparatus of claim 54, further comprising a non-analyte detection apparatus in data communication with the data processor apparatus;
wherein the autonomous operation further comprises:
collection of non-analyte signals generated by the non-analyte detection apparatus over at least a portion of the period of time;
processing of at least a portion of the non-analyte signals to produce processed non-analyte signal data; and
storage of the processed non-analyte signal data in the data storage apparatus.

61. The implantable blood glucose sensor apparatus of claim 61, wherein the plurality of instructions are further configured to, when executed by the data processor apparatus, cause the implantable blood glucose sensor apparatus to:
after the period of time, establish wireless data communication with the receiver apparatus; and
based at least on part on the establishment of the wireless data communication, wirelessly transmit at least a portion the processed non-analyte signal data to the receiver apparatus.

62. An implantable computerized apparatus configured to generate blood glucose data from signals collected from at least one blood glucose detector apparatus, the implantable computerized apparatus comprising:
wireless interface apparatus configured for wireless data communication with one or more computerized devices;
data processor apparatus in data communication with the wireless interface apparatus;
data storage apparatus in data communication with the data processor apparatus, the data storage apparatus having at least one computer program stored thereon, the at least one computer program comprising a plurality of instructions, the plurality of instructions configured to, when executed by the data processor apparatus, cause the implantable computerized apparatus to:
access processed blood glucose signal data stored at the data storage apparatus;
utilize the processed blood glucose signal data to generate data indicative of blood glucose level;
store the data indicative of blood glucose level at the data storage apparatus;
subsequent to the storage of the data indicative of blood glucose level, identify that at least one of the one or more computerized devices is communicative with the wireless interface apparatus; and
based at least in part on the identification, transmit at least a portion of the data indicative of blood glucose level to the at least one of the one or more computerized devices.

63. The implantable computerized apparatus of claim 62, wherein the plurality of instructions are further configured to, when executed by the data processor apparatus, cause the implantable computerized apparatus to:
utilize the data indicative of blood glucose level and previously generated data indicative of blood glucose level to generate one or more of trend data or rate of change (ROC) data, the previously generated data indicative of blood glucose level stored in the data storage apparatus;
store the one or more of the trend data or the ROC data at the data storage apparatus; and
based at least in part on the identification, transmit at least a portion of the one or more of the trend data or the ROC data to the at least one of the one or more computerized devices.

64. The implantable computerized apparatus of claim 62, wherein:
the at least one of the one or more computerized devices comprises an implantable medicant delivery apparatus; and
the transmission of the at least portion of the data indicative of the blood glucose level measurement comprises transmission of the at least portion of the data indicative of the blood glucose level to the implantable medicant delivery apparatus, the implantable medicant delivery apparatus configured to utilize the at least portion of the data indicative of the blood glucose level for generation of medicant dosing data or control signal data.

65. The implantable computerized apparatus of claim 62, wherein the plurality of instructions are further configured to, when executed by the data processor apparatus, cause the implantable computerized apparatus to:
based at least in part on the identification, wirelessly receive configuration data from the at least one of the one or more computerized devices, the configuration data relating to at least one aspect of a configuration of the implantable computerized apparatus; and
alter the configuration of the at least one aspect based at least on the received configuration data.

66. The implantable computerized apparatus of claim 62, wherein the plurality of instructions are further configured to, when executed by the data processor apparatus, cause the implantable computerized apparatus to:
determine that one or more criteria for calibration of the at least one blood glucose detector apparatus are met;
based at least in part on the determination, generate data configured to request calibration; and
based at least in part on the identification, transmit the data configured to request calibration to the at least one of the one or more computerized devices.

67. A method of operating an implanted analyte sensor apparatus, the implanted blood glucose sensor apparatus comprising at least one analyte detector, data storage apparatus, and wireless interface apparatus, the wireless interface apparatus configured for data communication with at least an external receiver apparatus, the method comprising:
operating the implanted analyte sensor apparatus for a period of time without data communication with the receiver apparatus, the operating comprising at least: (i) collecting signals from the at least one analyte detector over the period of time, (ii) processing at least a portion of the signals from the at least one analyte detector to generate processed analyte signal data, (iii) utilizing the processed analyte signal data to calculate blood analyte level data, the blood analyte level data relating to the period of time, and (iv) storing the blood analyte level data at the data storage apparatus;
after the period of time, identifying that communication with the receiver apparatus is enabled; and
based at least in part on the identifying enabled communication with the receiver apparatus, wirelessly transmitting at least a portion of the blood analyte level data to the receiver apparatus.

68. The method of claim 67, wherein:
implanted analyte sensor apparatus further comprises at least one non-analyte detector; and
the operating further comprises: (i) collecting signals from the at least one non-analyte detector, (ii) processing at least a portion the signals from the at least one non-analyte detector to generate processed non-analyte signal data, and (iii) correlating the processed non-analyte signal data with the blood analyte data.

69. The method of claim 67, wherein:
the wireless interface apparatus is further configured for data communication with a calibration device; and
the method further comprises:
identifying that communication with the calibration device is enabled; and
based at least in part on the identifying the enabled communication with the calibration device, wirelessly receiving calibration data from the calibration device.

70. The method of claim 69, wherein the utilizing the processed analyte signal data to calculate blood analyte level data at least in part comprises utilizing the calibration data to calculate calibrated blood analyte level data.

71. The method of claim 67, wherein the operating the implanted analyte sensor apparatus for the period of time without communication with the receiver apparatus further comprises:
utilizing the blood analyte level data and a plurality of time-stamps associated therewith to determine one or more of trend data or rate of change (ROC) data; and
storing the one or more of trend data or ROC data at the data storage apparatus.

72. The method of claim 71, further comprising, based at least in part on the identifying enabled communication with the receiver apparatus, wirelessly transmitting at least a portion of the the one or more of trend data or ROC data to the receiver apparatus.

73. The method of claim 67, further comprising:
determining that one or more criteria for calibration of the at least one analyte detector are met;
based at least in part on the determining, generating data configured to request calibration; and
based at least in part on the identifying enabled communication with the receiver apparatus, wirelessly transmitting the data configured to request calibration to the at least one of the one or more computerized devices
